(19) United States
(12) Patent Application Publication
(10) Pub. No.: US 2021/0000975 A1
(43) Pub. Date: Jan. 7, 2021

(54) MODRNA ENCODING SPHINGOLIPID-METABOLIZING PROTEINS

(71) Applicant: ICAHN SCHOOL OF MEDICINE AT MOUNT SINAI, New York, NY (US)

(72) Inventors: Efrat ELIYAHU, New York, NY (US); Lior ZANGI, New York, NY (US); Adam VINCEK, New York, NY (US); Yoav HADAS, New York, NY (US)

(21) Appl. No.: 16/978,024

(22) PCT Filed: Mar. 7, 2019
(86) PCT No.: PCT/US2019/021218

§ 371 Date: Sep. 3, 2020

Related U.S. Application Data
(60) Provisional application No. 62/639,691, filed on Mar. 7, 2018.
(60) Provisional application No. 62/692,185, filed on Jun. 29, 2018.

(51) Int. Cl. A61K 48/00; A61K 31/7088

(52) U.S. Cl. CPC A61K 48/0016; A61K 31/7088; C12Y 207/01091; C12Y 305/01023; A61K 48/005

(57) ABSTRACT

The present disclosure pertains to the use of a modified RNA (modRNA) that encodes a sphingolipid-metabolizing protein such as acid ceramidase to achieve expression of the sphingolipid-metabolizing protein in a mammalian cell or group of cells. Expression of the protein from the (modRNA) reduces high levels of ceramide in the cell that lead to cell death or senescence.

### CROSS-REFERENCE TO RELATED APPLICATIONS

This application claims the benefit of U.S. provisional application number 62/639,691 filed on Mar. 7, 2018 and U.S. provisional application number 62/692,185 filed Jun. 29, 2018.

### SEQUENCE LISTING

The instant application contains a Sequence Listing, created on Mar. 1, 2018; the file, in ASCII format, is designated 3710039AWO_sequencelisting_ST25.txt and is 32.4 kilobytes in size. The file is hereby incorporated by reference in its entirety into the instant application.

### TECHNICAL FIELD

The present disclosure relates generally to the use of sphingolipid-metabolizing proteins to improve the robustness and survival of cells. Specifically, expression of sphingolipid metabolizing proteins from modRNA inhibits cell death, promotes normal cellular function, and prolongs survival of cells.

### BACKGROUND OF THE DISCLOSURE

Different types of stress can initiate a transduction signal that leads to cell death. The pathway involves sphingolipid metabolism, mainly an increase in the level of ceramide that can lead to cell death. Previous methods to balance the level of ceramide in order to prevent the initiation of the cell death pathway have focused on ceramide synthesis.

One example of the application of the present technology is to improve survival of oocytes and embryos for use in reproductive technologies such as in vitro fertilization (IVF). Oocytogenesis, the process by which primary oocytes are formed, is complete either before or shortly after birth and no additional primary oocytes are created thereafter. In humans, therefore, primary oocytes reach their maximum development at approximately 20 weeks of gestational age.

Under normal physiological conditions, 85-90% of these oocytes succumb to apoptosis at some point during fetal or postnatal life; at birth approximately 1-2 million oocytes remain of the approximately seven million formed. Moreover, during a female's reproductive life, ovulated oocytes undergo molecular changes characteristic of apoptosis unless successful fertilization occurs. Clinically, when the remaining oocyte reserve has been exhausted (on average, this occurs in women around age 50), menopause ensues as a direct consequence of ovarian senescence.

For women of advanced reproductive age who still wish to become pregnant, the promise of in vitro fertilization (IVF) can provide a solution to diminished oocyte reserve. A major challenge of assisted reproduction technologies (ARTs), however, is to mimic the natural environment required to sustain oocyte and embryo survival in vitro.

There are several studies that support association of ceramide with cellular and organismal aging, which among other things, impacts reproduction. Ceram ides are bioactive lipids that mediate cell proliferation, differentiation, apoptosis, adhesion and migration. High levels of cellular ceram ides can trigger apoptosis whereas ceramide metabolites, such as ceramide 1 phosphate and sphingosine 1 phosphate, are associated with cell survival and proliferation.

The ability to promote cell survival may also be important therapeutically. For example, in acute myocardial infarction (MI), the level of lipids in the patient's blood can serve to predict the risk for complication. In particular, high levels of ceram ides have been associated with a higher probability of recurring events and mortality.

Methods for delivery of acid ceramidase as an mRNA to express the sphingolipid-metabolizing protein have been explored. The use of unmodified exogenous RNA as a gene delivery method however, is ineffective due to its instability outside the cell and the strong innate immune response it elicits when transfected into cells.

Therefore, what is needed is a RNA delivery method that can achieve short term expression of a sphingolipid-metabolizing enzyme in cells to inhibit cell death, initiate survival and rescue cells from senescence, thereby promoting cell quality and cell survival.

### SUMMARY OF THE DISCLOSURE

The disclosed technology is based on the delivery and use of sphingolipid-metabolizing protein to modulate the fate of cells following a stress-related event and during aging. The present disclosure manipulates the ceramide signal transduction pathway to provide a method for inhibiting cell death and/or cell senescence, initiating cell survival and prolonging the life span of cells cultured in vitro or in vivo by administration of modified mRNAs (modRNA) that encode sphingolipid-metabolizing proteins.

In one aspect, the disclosure relates to a method to inhibit cell death and/or cell senescence and improve survival of a cell or group of cells, the method comprising administering to said cell or group of cells a modified RNA (modRNA) that encodes a sphingolipid-metabolizing protein. In some embodiments the sphingolipid-metabolizing protein is selected from the group consisting of (1) ceramidase (2) sphingosine kinase (SPHK), (3) sphingosine-1-phosphate receptor (S1PR). In some embodiments, the method involves contacting the cells or group of cells with a combination of modRNAs that encode (1), (2) and (3). In one embodiment, administering is by contacting said cell or group of cells with the modRNA for a period of time sufficient for the modRNA or plurality of modRNAs to be translated by the cells into ceramidase, SPHK, and/or S1PR. In another embodiment, administration is by injection of the modRNA into the cell, group of cells or tissue/organ.

In one embodiment, in addition to damage the cells may have sustained as the result of oxidative stress, cells that are undergoing or have undergone a stress-related event such as ischemia, reperfusion injury or myocardial infarction may benefit from said method.

Cells contacted with the modRNA are mammalian cells and may include without limitation cardiac cells, for example, cardiomyocytes, muscle cells, skin cells, hair cells of the ear, eye cells, gametes, oocytes, sperm cells, zygotes, and embryos.

In a related aspect, the disclosure relates to a method to improve the robustness and quality of oocytes and/or embryos in vitro, comprising contacting said oocytes or embryos with (1) modRNA that encodes ceramidase, (2) modRNA that encodes sphingosine kinase (SPHK), (3) modified RNA (modRNA) that encodes sphingosine-1-phosphate receptor (S1PR) or any combination of (1), (2), and (3).

In yet another related aspect, the disclosure relates to a composition comprising one or more modRNAs that encode ceramidase, modRNAs that encode sphingosine kinase (SPHK), and modRNAs that encode sphingosine-1-phosphate receptor (S1PR).

In one embodiment the modRNA encodes a ceramidase selected from acid ceramidase, neutral ceramidase and basic ceramidase.

In one embodiment the modRNA encodes acid ceramidase and has the oligonucleotide sequence of SEQ ID NO: 1. In another embodiment, the modRNA encoding AC has the oligonucleotide sequence of SEQ ID NO: 6. In another embodiment, the cells are contacted with a modRNA that encodes sphingosine kinase (SPHK) having the oligonucleotide sequence of SEQ ID NO: 2. In another embodiment, the sphingolipid metabolizing molecule is S1PR and the oligonucleotide encoding it has the sequence SEQ ID NO: 3.

In one aspect, the present disclosure relates to a method to improve quality/survival of cells comprising contacting said cells with a (1) modRNA that encodes ceramidase, (2) modRNA that encodes sphingosine kinase (SPHK), (3) modified RNA (modRNA) that encodes sphingosine-1-phosphate receptor (S1PR) or any combination of (1), (2), and (3).

Compositions comprising any combination of modRNAs that encode (1) a ceramidase, (2) sphingosine kinase (SPHK), (3) sphingosine-1-phosphate receptor (S1PR) are also encompassed by the present disclosure.

The disclosure also relates to the use of a composition comprising (1) a modRNA that encodes a ceramidase; (2) a modRNA that encodes sphingosine kinase (SPHK), (3) a modRNA that encodes sphingosine-1-phosphate receptor (SIPR) or a combination of (1), (2), or (3) to prevent apoptotic cell death in cells and promote survival.

### BRIEF DESCRIPTION OF THE DRAWINGS

FIGS. 1A-1E show the characterization of cell death dynamics and sphingolipids metabolizing enzymes expression in mouse heart after MI. Hearts were harvested from sham operated mice or 4 hours 1, 2, 4 and 28 days post MI. A) TUNEL stain was used to assess DNA fragmentation in cardiac cells in non-treated, 1, 2, 4 and 28 days post MI. Troponin-I immunostaining was used to distinguish between cardiomyocytes and non-cardiomyocytes. B) Dendogram of Sphingolipids signaling pathway transcriptome in sham hearts, 4 h and 24 h post ligation. C) Acid Ceramidase (AC), Sphk1 and S1PR2 mRNA levels relative to 18s rRNA was assessed in LV in early stages of MI development by quantitative PCR D) Protein levels of AC and Sphk1 was assessed in LV in early stages of MI development by western-blot. E) AC activity in LV after MI in early stages of MI development.

FIGS. 2A-2C show the effects of sphingolipids metabolizing enzymes on anoxia induced apoptosis in neonate Rat cardiomyocytes. Primary cardiomyocytes were isolated from 2-3 days old Rats hearts. 2 days after the isolation the cells were transfected with modRNA encoding for AC, Sphk1 and S1PR2 A) 18 h post transfection the cells were fixed and immunostained to confirm a successful overexpression of the protein or B) transferred to anoxic condition for 48 h and then stained with Annexin 5 and DAPI to assess the Effects of individual genes or C) genes combinations on apoptosis level of cardiomyocytes.

FIGS. 3A-3B show the effects of sphingolipids metabolizing enzymes on apoptosis in LV of mice hearts 48 h post MI. modRNA encoding for Luc, AC, Sphk1 and S1PR2 were injected to mice hearts at time of MI induction or to sham hearts. A) 24 h post injection to sham hearts the hearts were harvested, fixed and immunostained to confirm a successful overexpression of the proteins or B) 48 h post injection to infarcted hearts the hearts were harvested, fixed and stained for TUNEL to assess the level of apoptosis in the LV after injecting single gene or genes combinations to the myocardium.

FIGS. 4A-4G show the effects of AC, Sphk1 and a combination of AC and Sphk1 on heart function and remodeling post MI. modRNA encoding for Luc, AC, Sphk1 or a combination of AC and Sphk1 were injected to mice hearts at time of MI induction. % fractioning shortening LVIDd and LVIDs was measured 2 days and 28 days post MI. on the 29th day post MI the hearts were harvested and fixed for scar size measurements. A) % fractioning shortening 28 days post MI. B) Delta of % fractioning shortening 28 days−2 days post MI. C) Left ventricular internal dimension-diastole (LVIDd) D) Left ventricular internal dimension-systole (LVIDs) E) Masson's trichrome stain and F) % scar area of left ventricle area. G) Survival curve 90 days after MI.

FIGS. 5A-5E show the characterization of cell death dynamics and sphingolipids metabolizing enzymes expression in mouse heart after MI. A) Dendogram of Sphingolipids metabolism genes transcriptome in sham hearts, 4 h and 24 h post ligation. B) Volcano plots of Sphingolipids metabolism genes transcriptome and Sphingolipids signaling pathway transcriptome 4 h and 24 h. C) Protein levels of Pro caspase and cleaved caspase in sham hearts and 24 h post MI in LV. D) Protein levels of Sphk1 and B-Actin in sham hearts 4 h and 24 h post MI. E) Protein levels of S1PR2 and B-Actin in sham hearts 4 h and 24 h post MI.

FIGS. 6A and 6B A) AC, Sphk1 and S1PR2 overexpression in human HEK293 cells. B. AC overexpression in induced pluripotent stem cells derived CM and it effect on cell death after 48 h in anoxia.

FIGS. 7A-7C show the effect of AC overexpression on protein expression enzyme activity and apoptosis 24 h post MI. A) AC activity in mice LV 24 h post MI. B) Caspase3 expression and AC expression 24 h post MI in control mice or mice treated with 100 μg AC modRNA. C) Effects of AC overexpression on DNA fragmentation 24 h post MI

FIGS. 8A-8D show the effects of AC, Sphk1 and AC+Sphk1 combination on heart function and remodeling post MI. A) % fractional shortening of LV 2 days post MI. B) Correlation between scar size and % FS at day 28 post MI C) Cardiomyocytes area 28 days post MI as assessed by WGA stain D) Number of luminal structures in LV 28 days post MI as assessed by CD31 immunostaining.

FIGS. 9A-9D show heart function parameters including outliers. A) % fractional shortening of LV 2 days post MI. B) % FS change between 2 days post MI and 28 days post MI C) % scar size 29 days post MI and D) Left ventricle internal dimension systolic 28 days post MI.

FIG. 10 shows the effects of ACv2 overexpression on scar size after ischemia and reperfusion injury in the LV.

FIG. 11A-11D shows that AC, S1PR and GFP modRNA were successfully translated into a protein after modRNA delivery. (A) PN embryos were injected with 50 ng of AC ModRNA or S1P RModRNA, collected after 24 h (2 cell stage) Proteins were detected using western blot analysis. Western blot analysis was performed using (a) mouse anti-human AC IgG, revealing the human AC precursor (at 55 kDa); (b) mouse anti-human S1PR IgG; (c) Rabbit anti-human Actin IgG. (B) PN embryos were injected with 50 ng GFP ModRNA, and analyzed for GFP protein expression on day 4 by light (left panel) and fluorescent (right panel) microscopy. (C) Mouse sperm were incubated with 100 ng/μl naked GFP ModRNA for 1 h in 37° C. CO₂ incubator. Post incubation, sperm were analyzed for GFP protein expression by fluorescent microscopy. (D) Mouse sperm were incubated with 100 ng/μl naked GFP ModRNA for 1 h in 37° C. CO₂ incubator. Post incubation, sperm were incubated with C57BL/6 eggs for IVF. Embryos (blastocysts) at day 7 were analyzed for GFP protein expression by light (left panel) and fluorescent (right panel) microscopy.

FIGS. 12A-12F show that proteins were detected using western blot analysis. AC and SPHK1 modRNAs were successfully translated into protein after modRNA delivery, in vitro and in vivo. Cells and heart were transfected/injected with modRNA using RNAiMAX-lipofectamine then collected after 24 hours.

FIG. 13A-13B show the results of immunofluorescence analysis demonstrating expression of AC and SPHK1 modified mRNA in neonatal rat cardiomyocyte and mouse heart.

FIGS. 14A-14B show the results of immunofluorescence analysis demonstrating expression of GFP modified mRNA after injection into ovary in vivo. Mice were injected with transfection buffer (control) or GFP modRNA into the ovary. 24 hours post injection ovaries were removed, and analyzed by fluorescent microscopy for GFP expression. GFP is expressed in the ovary after direct injection.

FIGS. 15A-15H shows that AC modRNA prevent cell death in serum starvation MBD-mb-231 human breast cancer cell line model in vitro. Cells were transfected with modRNA using iMAX-lipofectamine, cultured for 48 hours and were analyzed by fluorescent microscopy. AC reduced apoptotic activation after delivery into breast cancer cell model in vitro.

FIGS. 16A and 16B show that AC modRNA delivery immediately after myocardial infarction, prevent apoptosis activation in vivo. (A) Mice were injected with Luc or AC modRNA and undergo MI. 24 hours post injury, hearts were removed, lysed and proteins were analyzed by western blot analysis (Control lane no MI). AC inhibited apoptosis evaluated by Caspase 3 expression. AC also can reduce TNF alpha when there is higher AC expression. (B) Mice were injected with Luc control or AC modRNA and undergo myocardial infarction. 8 hours post injury, hearts were removed, lysed and proteins were analyzed by western blot analysis. AC and SHPK1 inhibit the cleavage of PARP by kaspas3 during apoptosis.

FIGS. 17 shows the effect of pro-survival genes on anoxia induced apoptosis in neonatal rat CM.

FIG. 18 shows the effect of AC on apoptosis 2 days after permanent MI.

FIG. 19 shows that AC and SHPK1 mod RNA delivery, immediately after MI, reduce significantly heart cardiac scar size. Mice were injected with Luc control or AC modRNA and undergo MI, one month post injury, hearts were removed, perfused, fixed and stained for scar formation (Masson's trichrome staining) red indicates healthy tissue while blue indicates scarred tissue. AC or SPHK1 modRNA delivery significantly reduced heart scar size.

### DETAILED DESCRIPTION OF THE DISCLOSURE

All patents, published applications and other references cited herein are hereby incorporated by reference into the present application.

In the description that follows, certain conventions will be followed as regards the usage of terminology. In general, terms used herein are intended to be interpreted consistently with the meaning of those terms as they are known to those of skill in the art. Some definitions are provided purely for the convenience of the reader.

The term “cell or group of cells” is intended to encompass single cells as well as multiple cells either in suspension or in monolayers. Whole tissues also constitute a group of cells.

The term “cell quality” or “quality of a cell” refers to the level of cell viability, and cellular function of a cell as measured against a normal healthy cell of the same type with normal cell function and expected life span, the quality of cells that are programmed for survival but not for cell death. Embryo quality is the ability of an embryo to perform successfully in terms of conferring a high pregnancy rate and/or resulting in a healthy offspring and is assessed mainly by microscopic evaluation at certain time points following in vitro fertilization. Embryo profiling is the estimation of embryo quality by qualification and/or quantification of various parameters known to those of skill in the art including but not limited to number of pronuclei, cell number, cell regularity, degree of fragmentation. Estimations of embryo quality guides the choice in embryo selection in in vitro fertilization.

The term “inhibit” or “inhibition” when used in conjunction with senescence includes the ability of the sphingolipid-metabolizing proteins of the disclosure to reverse senescence, thereby returning to normal or near normal function.

The terms “stress”, “stress-related events” or “cellular-stress” refer to a wide range of molecular changes that cells undergo in response to environmental stressors, such as extreme temperatures, exposure to toxins, mechanical damage, anoxia, and noise.

The term “robustness” as it is used herein, refers to the quality or condition of being strong and in good condition.

Duration of expression can be tailored to the specific situation by choice of gene delivery method. The term “short term expression,” for example, refers to expression of the desired protein for a duration of several days rather than weeks. So, for example, the use of modRNA as a gene delivery method achieves transient expression of the selected sphingolipid-metabolizing protein for up to about 11 or 12 days. Quick, transient expression of short duration may be sufficient, for example, to extend survival and the quality of oocytes and embryos prior to IVF.

The term “modRNA” refers to a synthetic modified RNA that can be used for expression of a gene of interest. Chemical modifications made in the modRNA, for example substitution of pseudouridine for uridine, stabilize the molecule and enhance transcription. Additionally, unlike delivery of protein agents directly to a cell, which can activate the immune system, the delivery of modRNA can be achieved without immune impact. The use of modRNA for in vivo and in vitro expression is described in more detail in for example, WO 2012/138453.

### Shingolipid-Metabolizing Proteins

In one embodiment, a modRNA composition useful for the method of the present disclosure may include either individually or in different combinations modRNAs encoding the following sphingolipid-metabolizing proteins: ceramidase (acid, neutral or alkaline), sphingosine kinase (SPHK), and sphingosine-1-phosphate receptor (S1PR). In one embodiment, the sphingolipid-metabolizing protein is a ceramidase.

Ceramidase is an enzyme that cleaves fatty acids from ceramide, producing sphingosine (SPH), which in turn is phosphorylated by a sphingosine kinase to form sphingosine-1-phosphate (S1P). Ceramidase is the only enzyme that can regulate ceramide hydrolysis to prevent cell death and SHPK is the only enzyme that can synthesize sphingosine 1 phosphate (S1P) from sphingosine (the ceramide hydrolysis product) to initiate cell survival. S1PR, a G protein-coupled receptor binds the lipid-signaling molecule S1P to induce cell proliferation, survival, and transcriptional activation.

Presently, 7 human ceramidases encoded by 7 distinct genes have been cloned:
acid ceramidase (ASAH1)—associated with cell survival;
neutral ceramidase (ASAH2, ASAH2B, ASAH2C)—protective against inflammatory cytokines;
alkaline ceramidase 1 (ACER1)—mediating cell differentiation by controlling the generation of SPH and S1P;
alkaline ceramidase 2 (ACER2)—important for cell proliferation and survival; and
alkaline ceramidase 3 (ACER3).
   The nucleotide sequences for the coding sequences are shown below in Table 1.

The discovery by Kariko et al. (Incorporation of Pseudouridine Into mRNA Yields Superior Nonimunogenic Vector With Increased Translational Capacity and Biological Stability. Mol Ther. 2008; 16 (11): 1833-1840, incorporated herein by reference) that the substitution of uridine and cytidine with pseudouridine and 5-methylcytidine, respectively, drastically reduced the immune response elicited from exogenous RNA set the stage for a new kind of gene delivery, in which transient expression of therapeutic proteins is achieved.

Modified mRNA (modRNA) is a relatively new gene delivery system, which can be used in vitro or in vivo to achieve transient expression of therapeutic proteins in a heterogeneous population of cells. Incorporation of specific modified nucleosides enables modRNA to be translated efficiently without triggering antiviral and innate immune responses. In the present disclosure, modRNA is shown to be effective at delivering short-term robust gene expression of a “survival gene” and its use in the field of gene therapy is expanding. A stepwise protocol for the synthesis of modRNA for delivery of therapeutic proteins is disclosed in, for example, Kondrat et al. Synthesis of Modified mRNA for Myocardial Delivery. Cardiac Gene Therapy, pp. 127-138 2016, the contents of which are hereby incorporated by reference into the present disclosure.

The use of modRNA, a relatively nascent technology, has considerable potential as a therapy for disease. Delivery of a synthetic modified RNA encoding human vascular endothelial growth factor-A, for example, results in expansion and directed differentiation of endogenous heart progenitors in a mouse myocardial infarction model (Zangi et al. Modified mRNA directs the fate of heart progenitor cells and induces vascular regeneration after myocardial infarction. Nature Biotechnology 31, 898-907 (2013)). In another example, diabetic neuropathy may be lessened by the ability to deliver genes encoding nerve growth factor. Additionally, with the advent of genome editing technology, CRISPR/Cas9 or transcription activator-like effector nuclease (TALEN), transfection will be safer if delivered in a transient and cell-specific manner.

In one embodiment of the present method, the gene delivery molecule that encodes a sphingolipid-metabolizing protein is modRNA. While various gene delivery methods exist for achieving expression of an exogenous protein, for example, using plasm ids, viruses or mRNA, in certain situations modRNA offers several advantages as a gene delivery tool.

An advantage of gene delivery over protein is the ability to achieve endogenous expression of protein for a specific period of time and therefore extended exposure to the sphingo-lipid metabolizing enzyme.

One advantage of modRNA delivery is the lack of a requirement for nuclear localization or transcription prior to translation of the gene of interest. Eliminating the need for transcription of an mRNA prior to translation of the protein of interest results in higher efficiency in expression of the protein of interest.

Kariko et al. showed in 2008 that uridine replacement in mRNA with pseudouridine (hence the name modified mRNA (modRNA)) resulted in changes to the mRNA secondary structure that avoid the innate immune system and reduce the recognition of modRNA by RNase. In addition, these changes of nucleotides are naturally occurring in our body and lead to enhance translation of the modRNA compared to unmodified mRNA.

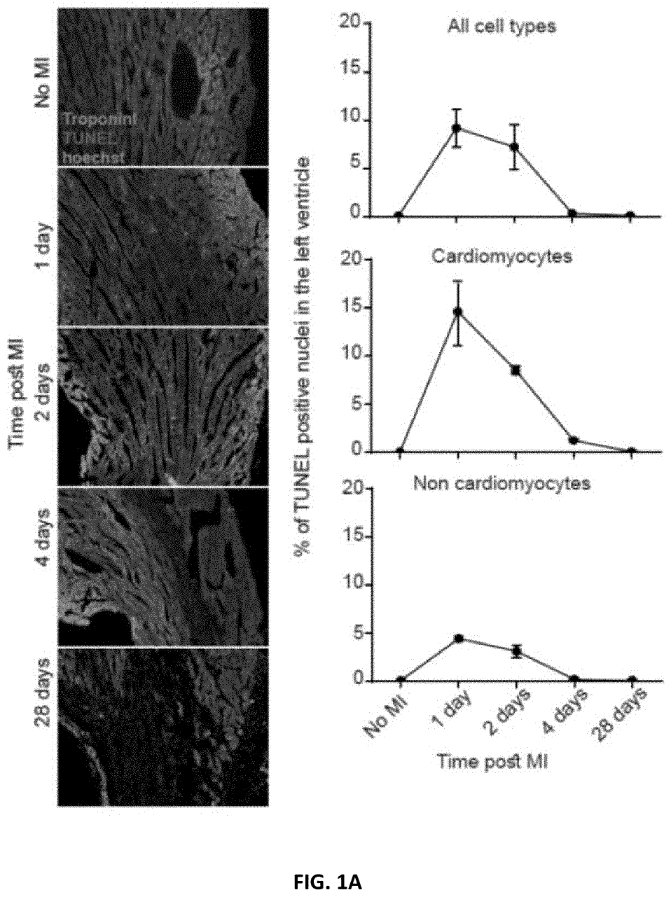

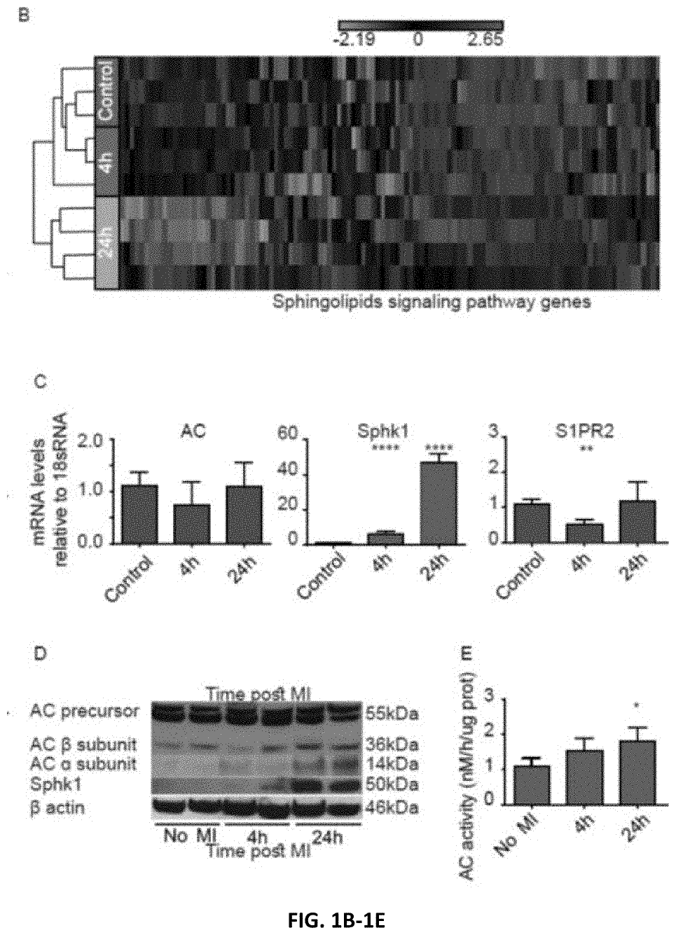

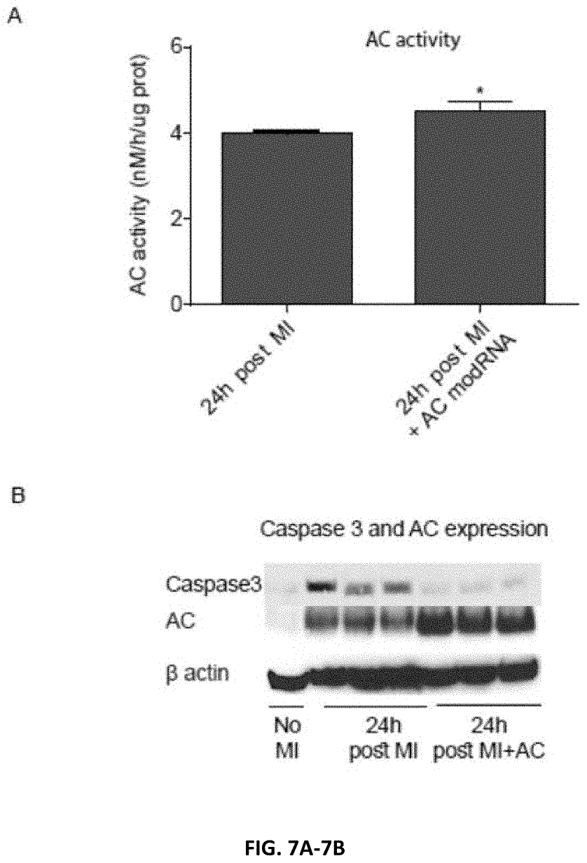

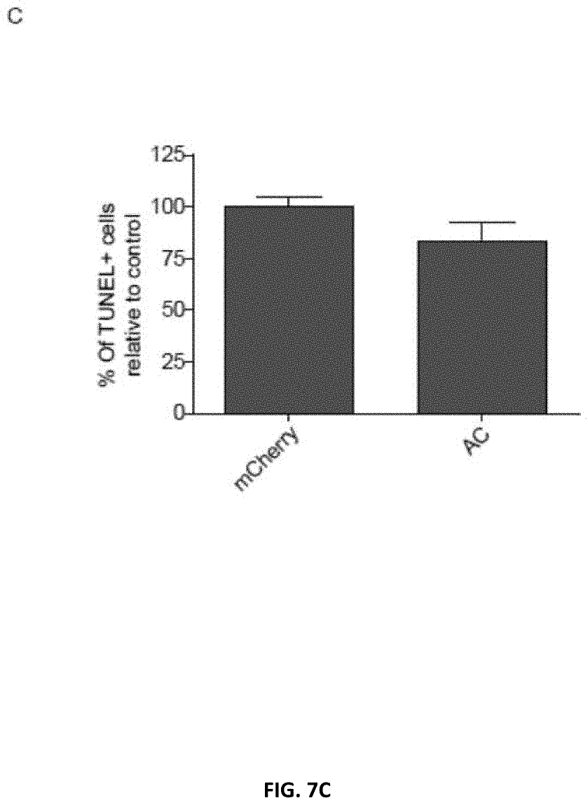

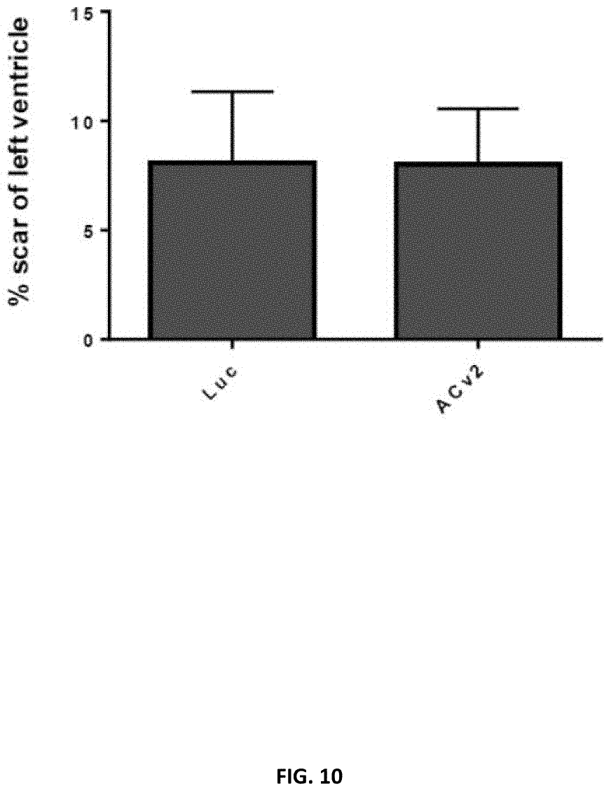

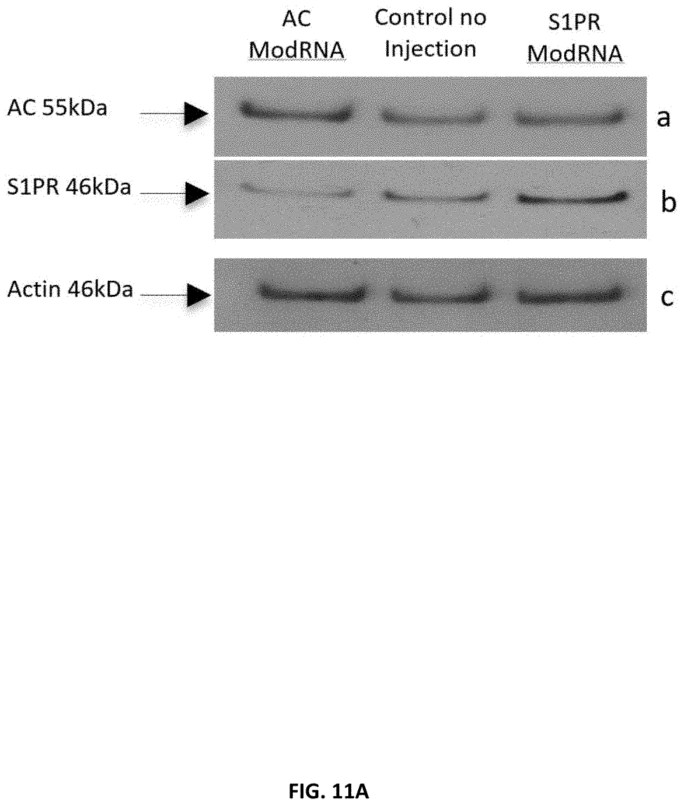

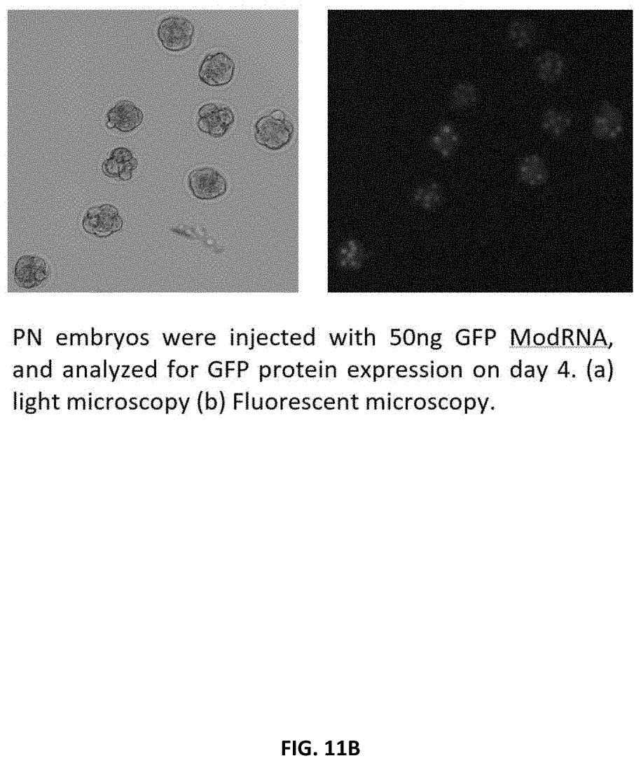

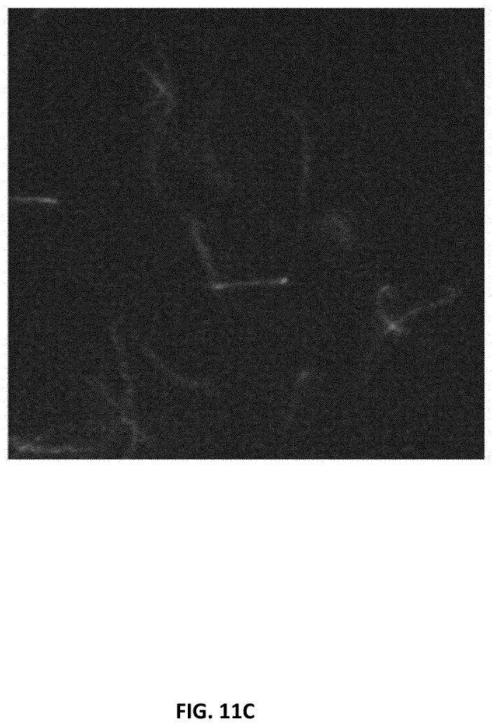

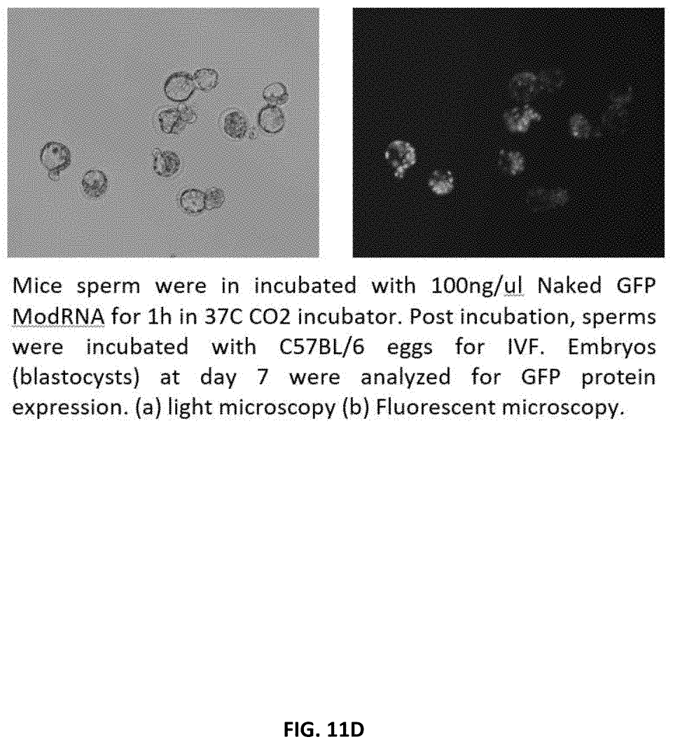

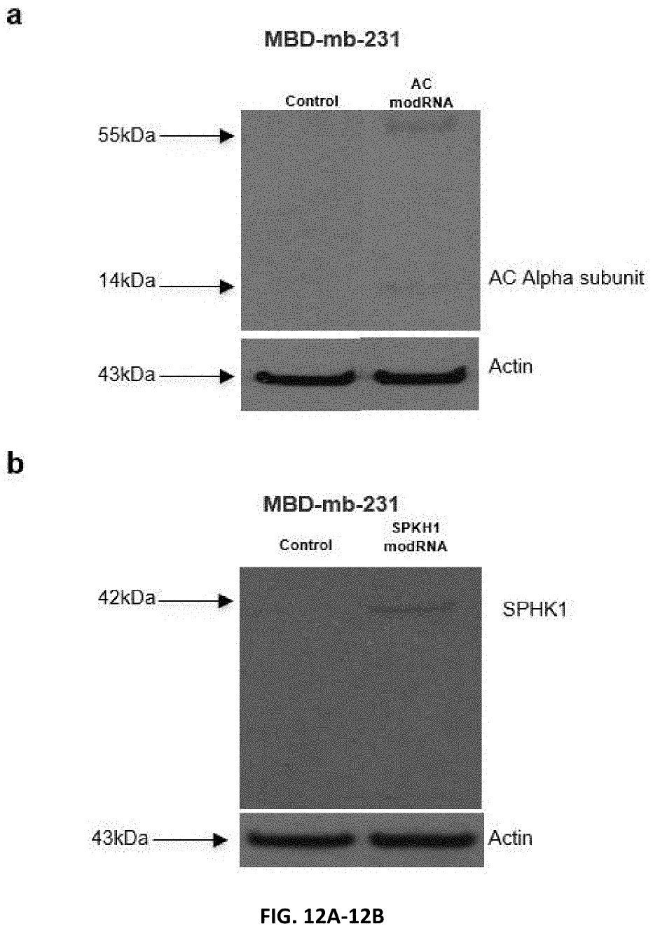

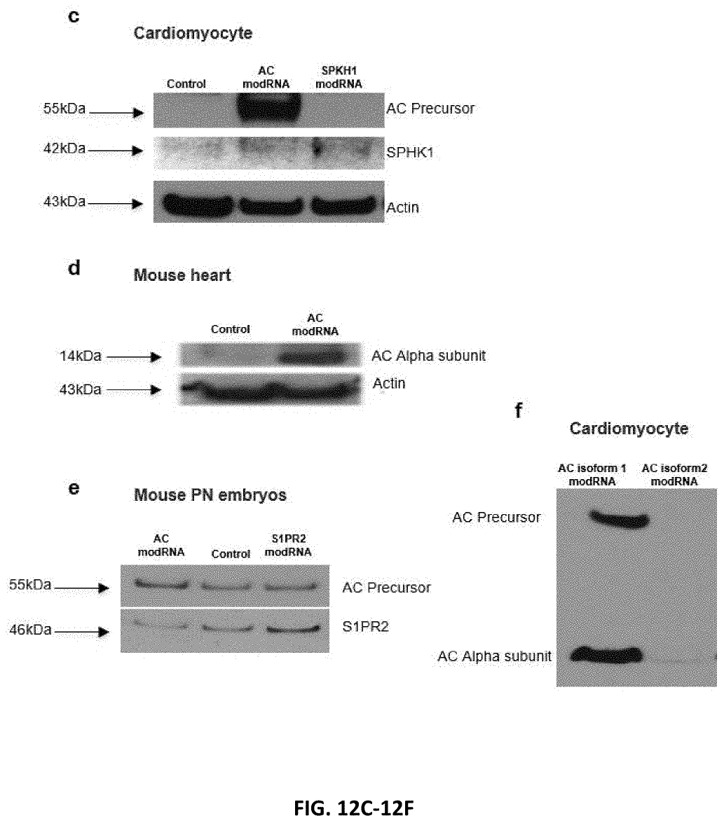

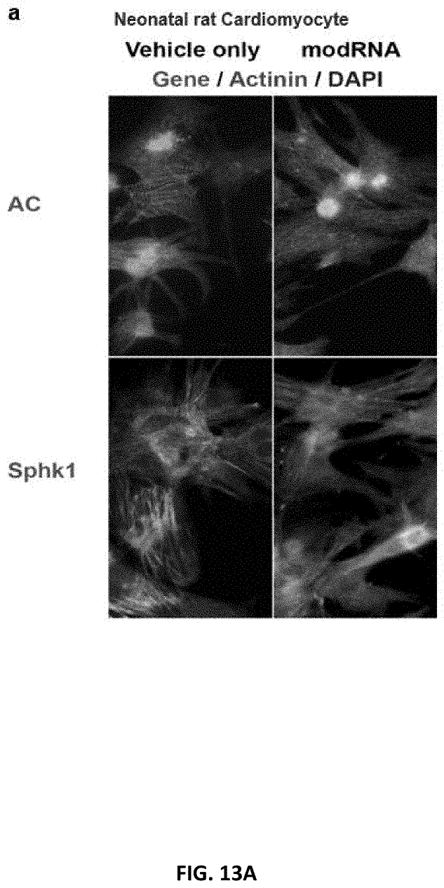

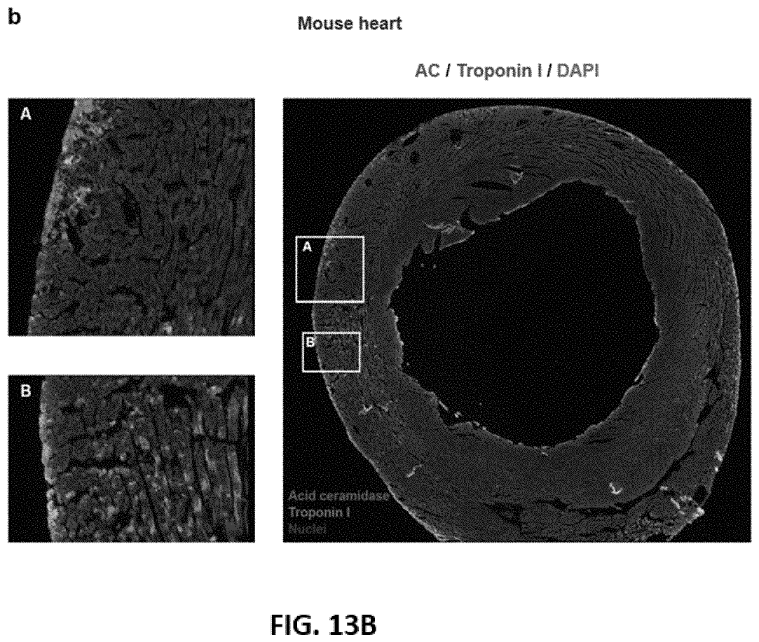

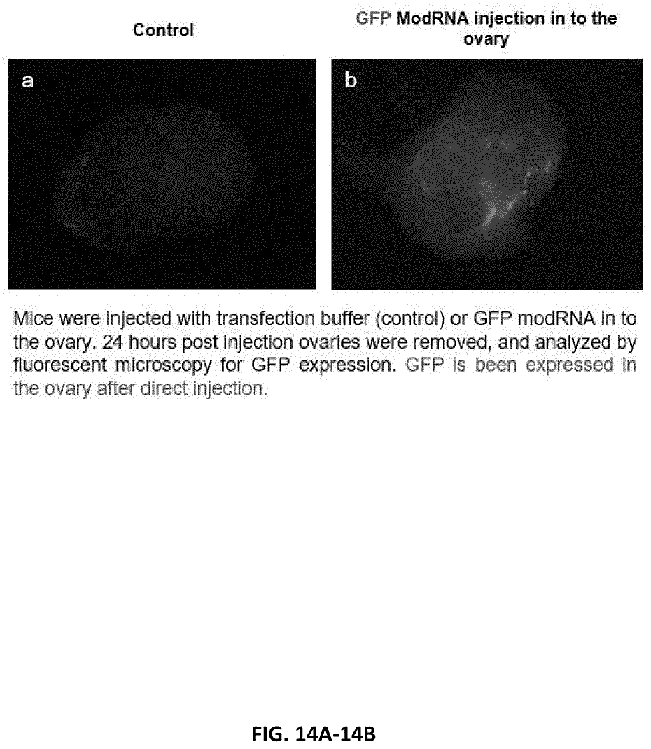

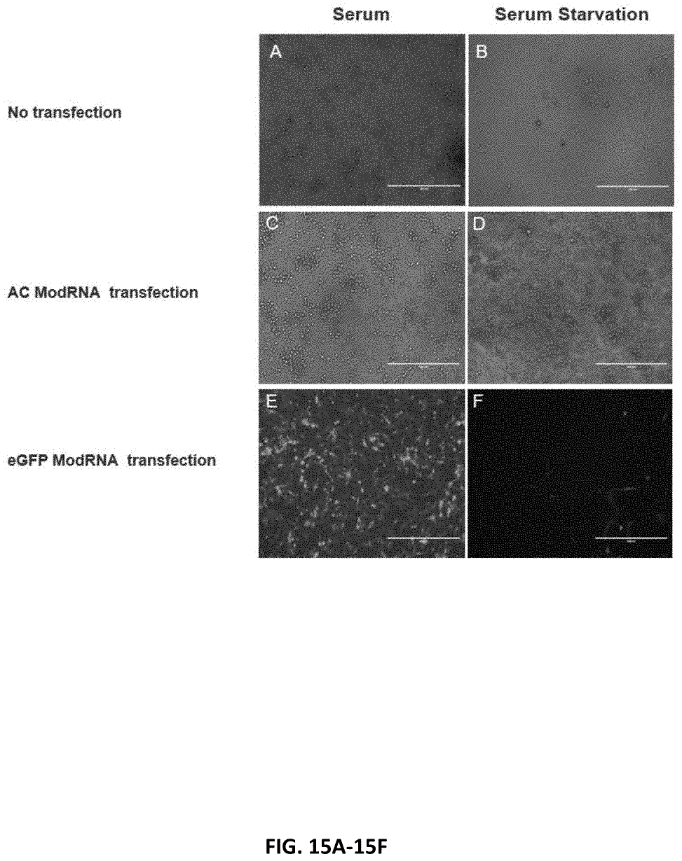

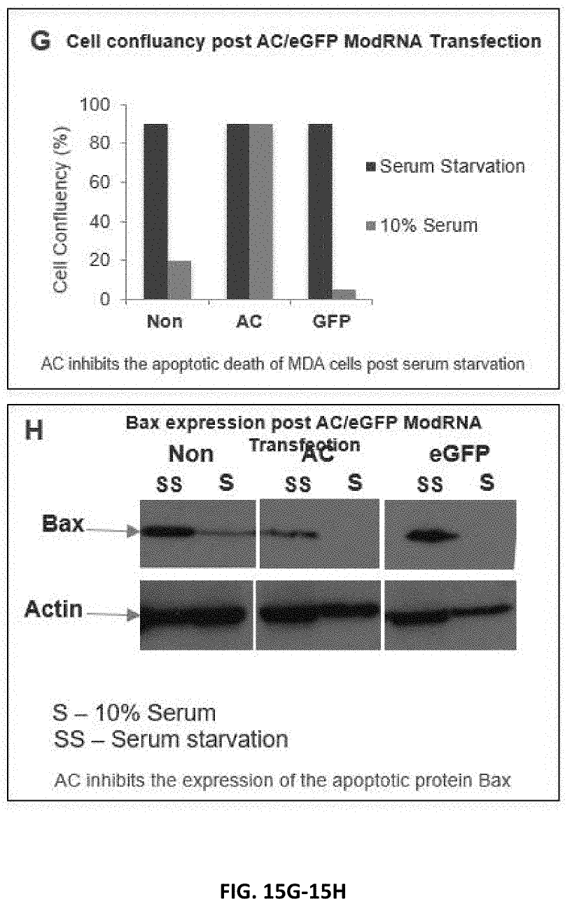

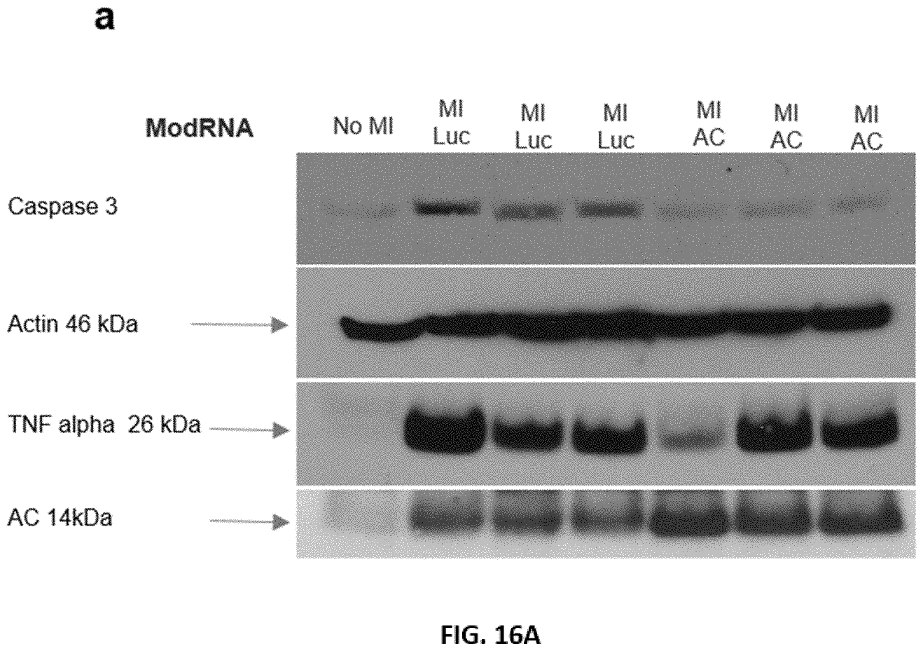

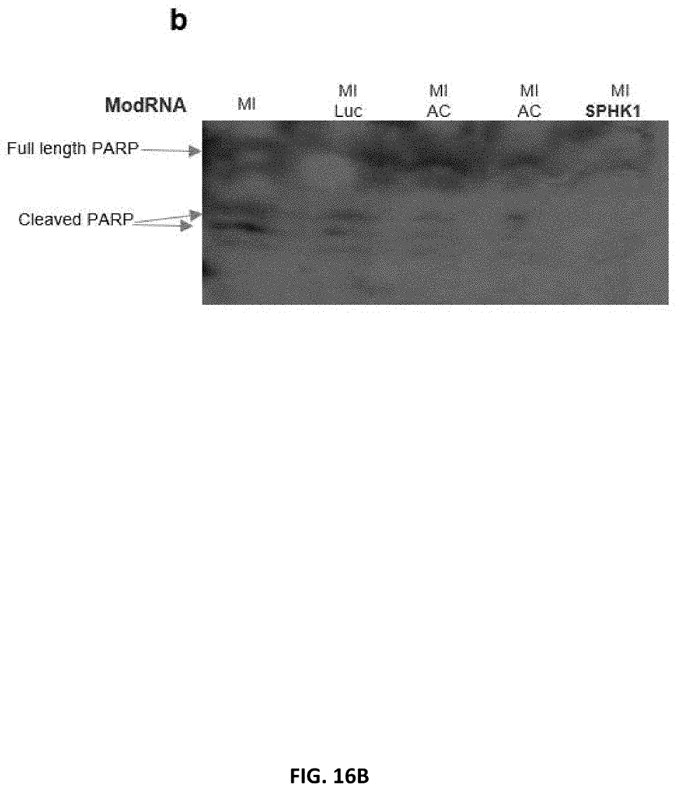

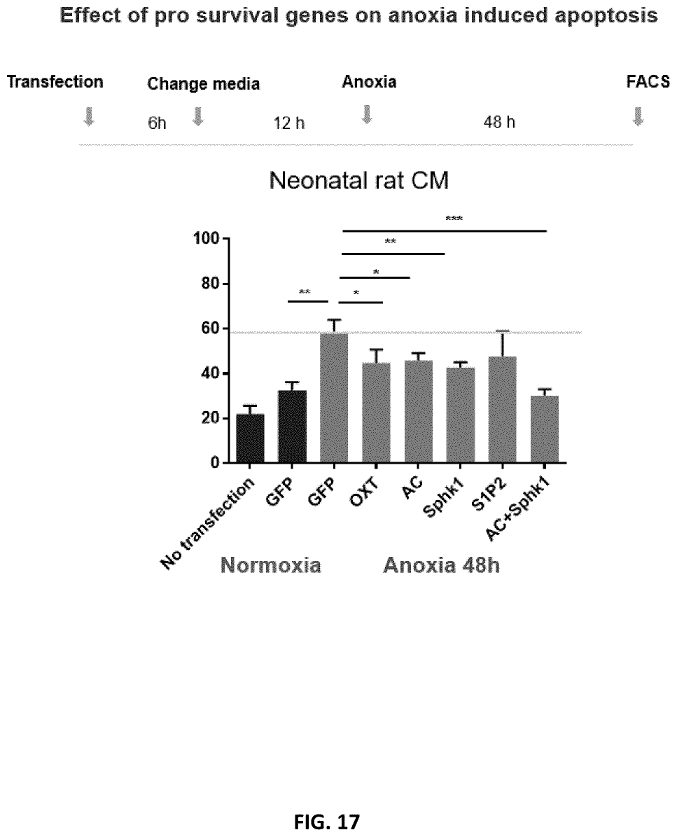

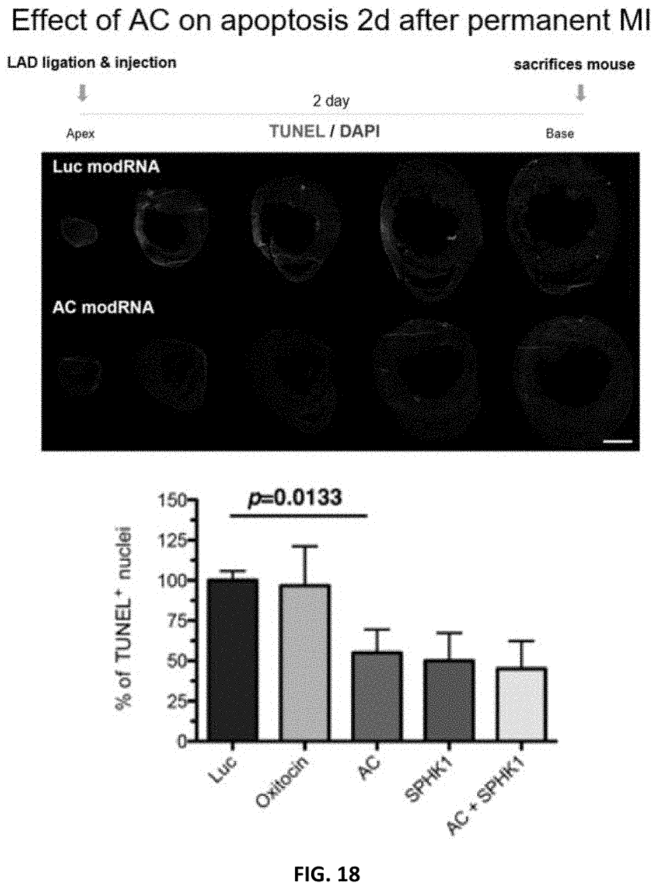

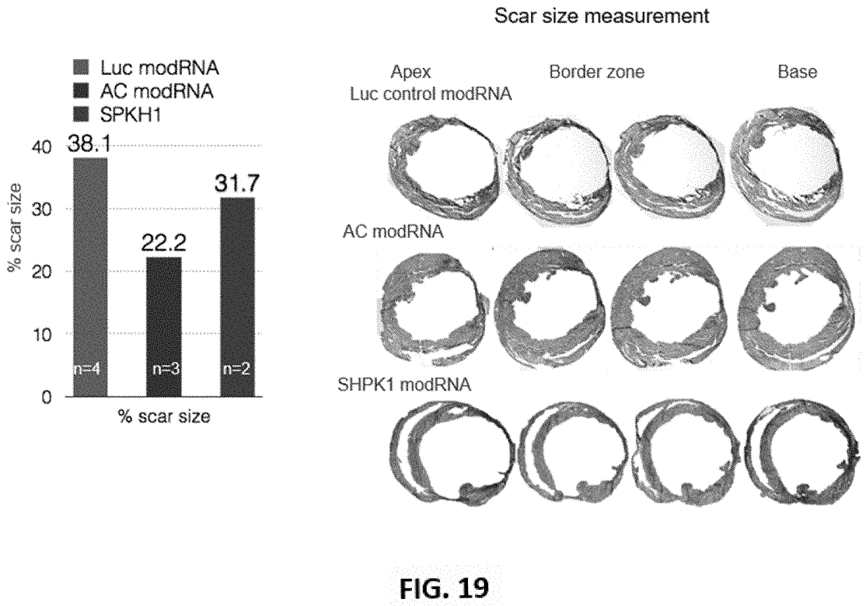

The present invention is based on the observation that administration of a modRNA “survival cocktail” comprising modRNAs that encode one or more sphingolipid-metabolizing proteins decreased the rate of apoptosis in vitro and in vivo in different cell types, tissue and embryos (FIGS. 1-19).

modRNA is a synthetic mRNA with an optimized 5′UTR and 3′UTR sequences, anti-reverse cup analog (ARCA) and one or more naturally modified nucleotides. The optimized UTRs sequences enhance the translation efficiency. ARCA increases the stability of the RNA and enhances the translation efficiency and the naturally modified nucleotides increase the stability of the RNA reduce the innate immune response of cells (in vitro and in vivo) and enhance the translation efficiency of the mRNA. This combination generates a superior mRNA that mediate a higher and longer expression of proteins with a minimal immune respond. Modified mRNA is a safe, local, transient, and with high expression gene delivery method to the heart. Kariko et al. have shown in 2008 that uridine replacement in mRNA with pseudouridine (hence the name modified mRNA (modRNA)) resulted in changes to the mRNA secondary structure that avoid the innate immune system and reduce the recognition of modRNA by RNase. In addition, these changes of nucleotides are naturally occurring in our body and lead to enhance translation of the modRNA compared to unmodified mRNA.

Since the modRNAs encode physiological enzymes, the expression of ceramidase should have little or no toxic effects. In addition, transfecting cells with ceramidase modRNA will increase the precursor (inactive form) of the enzyme that will allow autonomous control of the active ceramidase protein, which is required for survival. Furthermore, control of ceramide metabolism is the only known biological function of ceramidase; manipulation of ceramidase should not influence other cellular signaling. In addition, creation of a mouse model that continually overexpresses the AC enzyme (COEAC) in all tissues demonstrates a lack of toxicity or tumorigenesis effect by overexpression of AC.

Thirdly, messenger RNA modifications allow modRNA to avoid detection by the innate immune system and RNase. Based on that observation, modRNA can be used as a safe and effective tool for short-term gene delivery. Pharmacokinetics analyses of modRNA indicate a pulse-like expression of protein up to 7 days.

### Effect of Sphingolipid-Metabolizing Proteins on Cardiomyocytes

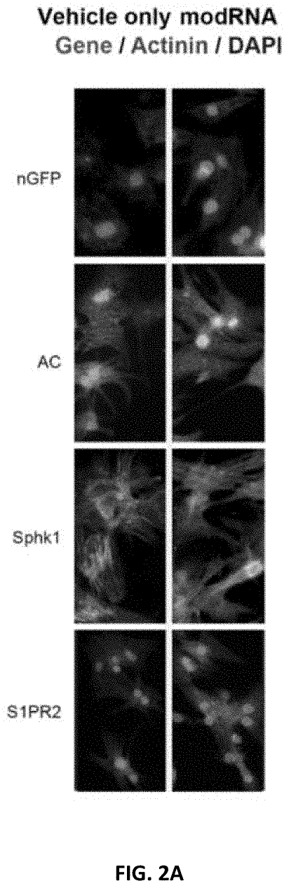

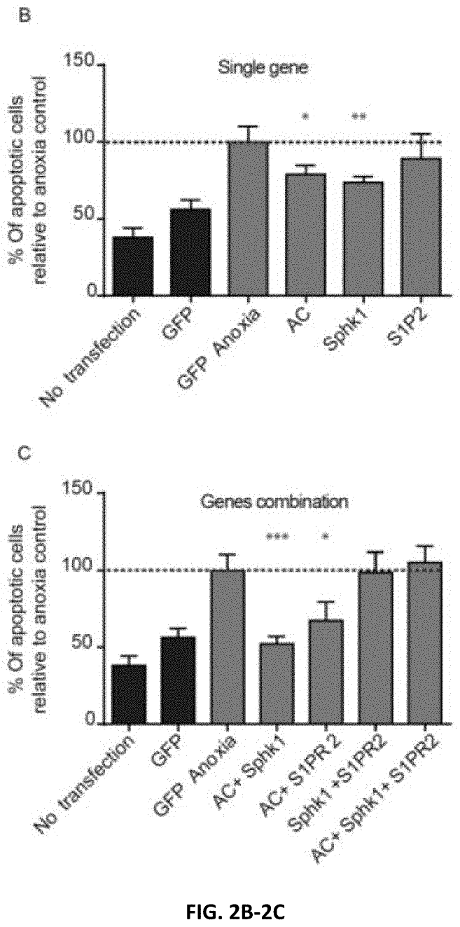

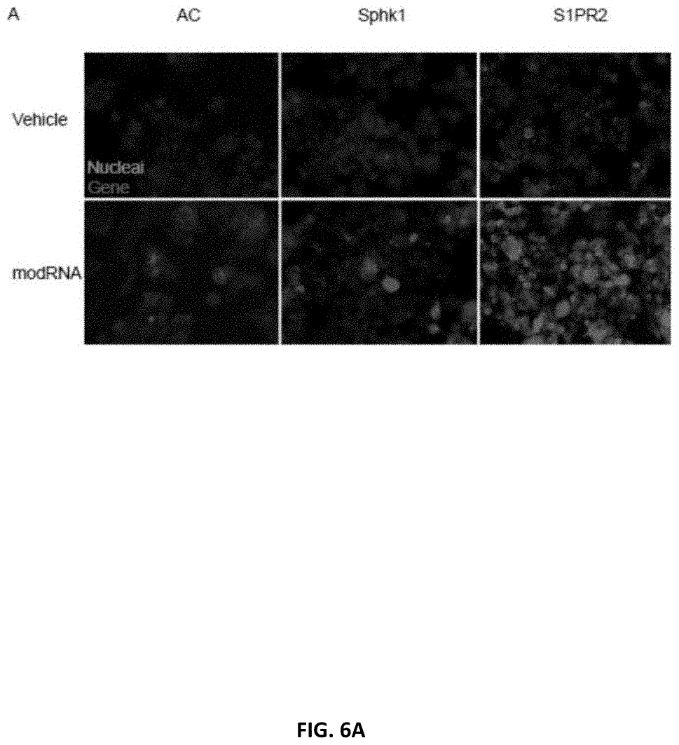

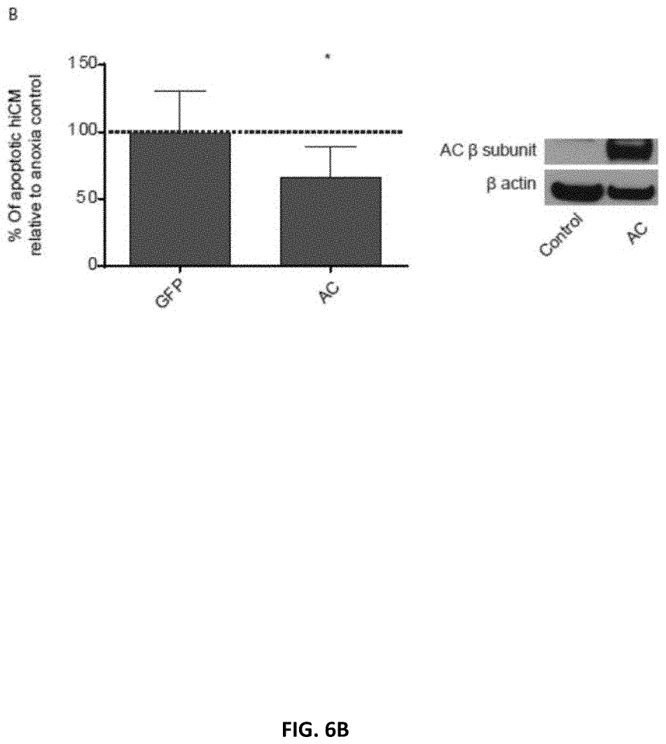

The effect of these genes on the viability of neonatal rat cardiomyocytes (nrCM) under anoxic conditions was examined. Synthetic modRNAs that encode human AC, Sphk1 and S1PR2 were used. The expression kinetics of proteins encoded by modRNA and its reduced immunogenicity (Sultana 2017) make modRNA an ideal vector to study the role of gene expression in acute conditions such as myocardial infarction. First, the effect of modRNA transfection on the expression levels of the target proteins in Hek293 cells (FIG. 6A) or nrCM (FIG. 2A) was checked. In both cases, the levels of the protein encoded by the transfect modRNA were elevated in the transfected cells compare to control cells. To induce apoptosis in nrCM the cells were transfer to anoxic condition 18 h after transfection. After 48 h in anoxia, there was an elevation of 44% in the number of apoptotic cells, however, overexpression of AC or Sphk1 reduced the level of apoptotic cells by 22% and 27% respectively compared to control (FIG. 2B). Overexpression of S1PR2 reduced the level of apoptosis by 10% however, this reduction was not statistically significant (FIG. 2B).

When the cells were transfected with a combination of genes an additive effect was observed. Overexpression of AC and Sphk1 reduced the number of apoptotic cells by 48% and overexpression of AC and S1PR2 together reduced apoptosis by 33%. Surprisingly, combining Sphk1 with S1PR2 or combining AC, Sphk1 and S1PR2 did not reduce the levels of apoptosis (FIG. 2C).

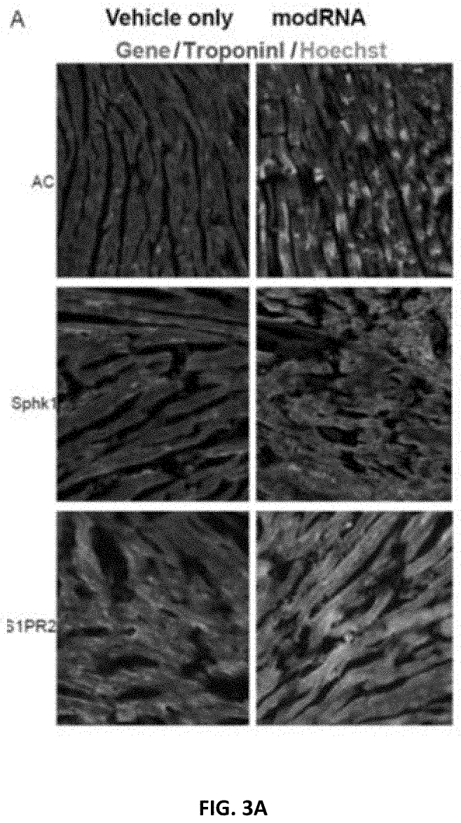

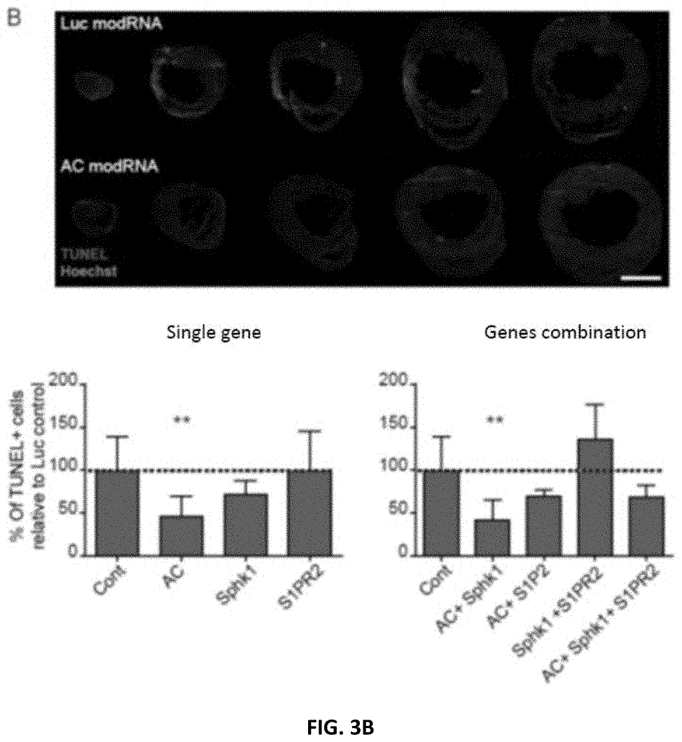

To study the effect of AC, Sphk1, and S1PR2 on cell death in LV after myocardial infarction, hearts were infarcted by ligation of the left anterior descending artery. Immediately after the LAD was ligated, 100 μg modRNA encoding to a control gene or gene of interest were injected to the myocardium of the left ventricle. After 48 h the hearts were harvests and the levels of DNA fragmentation was measured. Strikingly, overexpression of AC in the left ventricle immediately after LAD ligation reduced the number of cells with fragmented DNA in the left ventricle by 54% compare to hearts that were treated with Luc modRNA. Overexpression of Sphk1 reduced DNA fragmentation by 29% and S1PR2 did not prevent the fragmentation of DNA in the LV 48 h post-MI (FIG. 3B). When a combination of genes was injected to the LV immediately after LAD ligation, only the combination of AC and Sphk1 had a mild additive effect of 59% reduction. AC+S1PR2 reduce DNA fragmentation by 21% and AC+Sphk1+S1PR2 reduce DNA fragmentation by 22%. Unexpectedly, overexpression of Sphk1 and S1PR2 induced DNA fragmentation post MI by 30% compare to control (FIG. 3B).

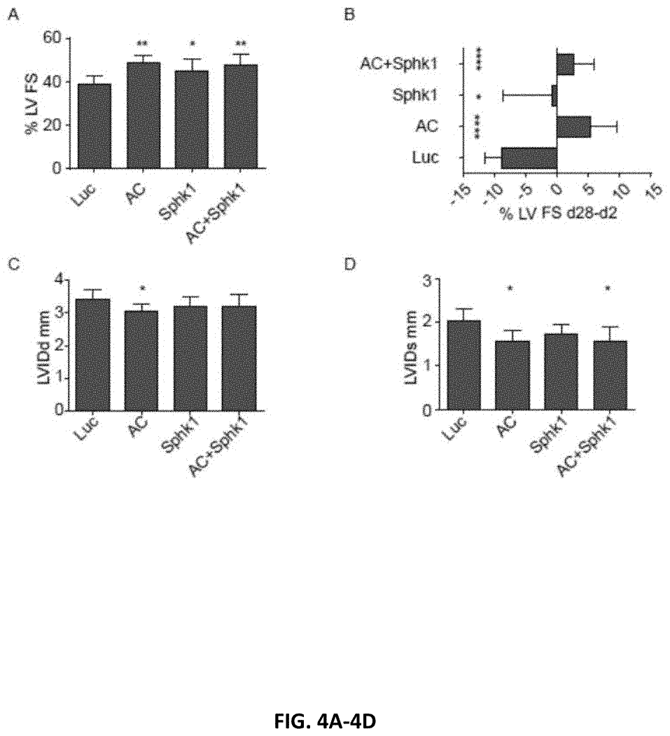

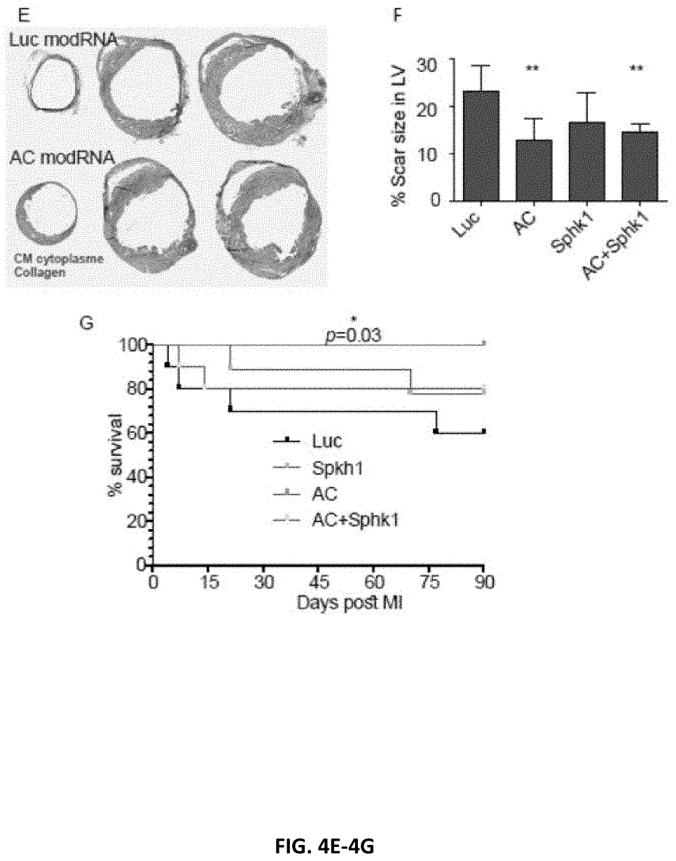

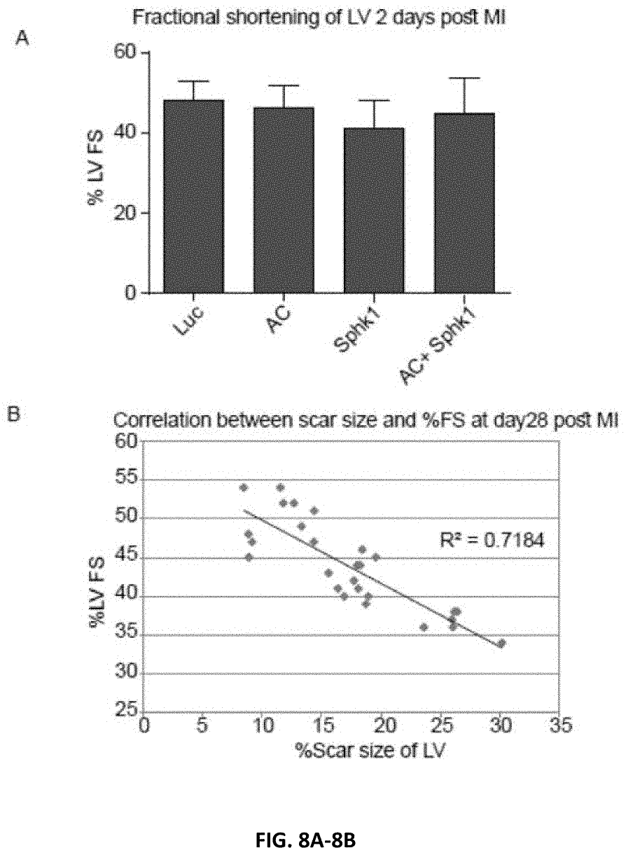

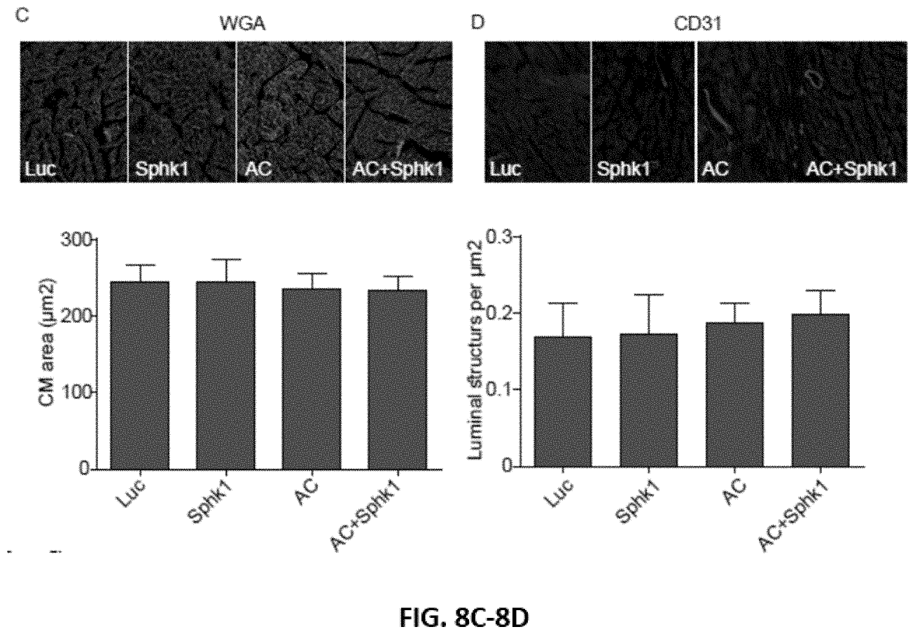

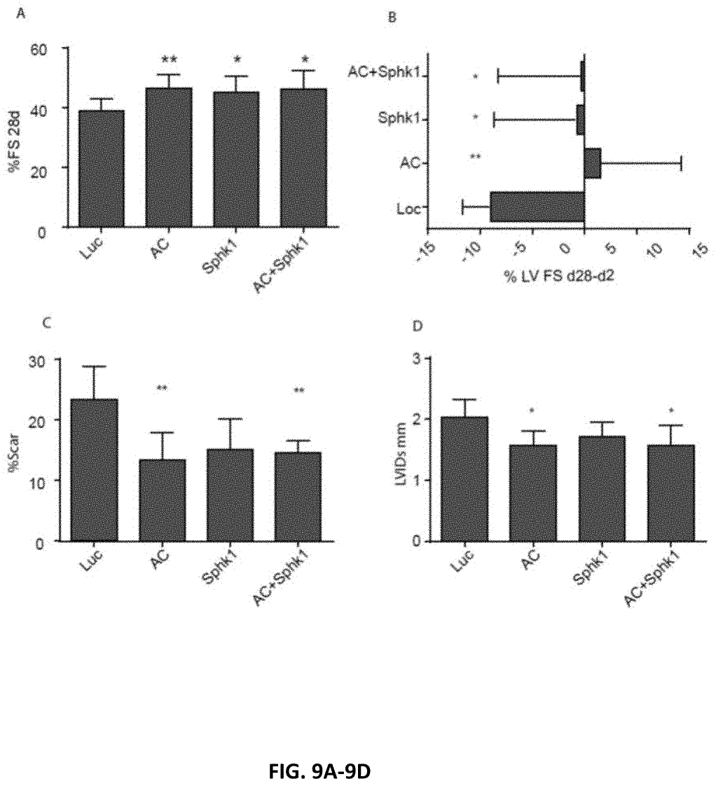

The beneficial effects of AC and Sphk1 and the additive effect of the combined expression of these two genes prompted us to study their effect on heart remodeling and function post MI. To this aim, we injected AC, Sphk1, AC+Sphk1 or Luc directly to the LV and compare the Left ventricular internal dimension-diastole (LVIDd), Left ventricular internal dimension-systole (LVIDs) and fractioning shortening % (% FS) at different time point post MI. At the end of the experiment (29 days post MI) the hearts were harvested and immunostained with WGA and CD31 to assess the average area of cardiomyocytes and the number of vessels in the LV. To measure the scar size, Masson's trichrome stain was performed on heart sections. Two days post-MI, there was no significant difference between the groups in all measured parameters (FIG. 8A). However, 28 days post MI % FS of LV in mice that were treated with AC Sphk1 or AC+Sphk1 were 46.4% 45% and 46.1% respectively compared to 38.8% in control mice (FIG. 9A). The LVIDs of mice treated with AC Sphk1 or AC+Sphk1 were lower than in control mice—1.65 mm, 1.72 mm, and 1.57 mm respectively compared to 2.02 mm in control LVIDd of treated mice was not significantly different than the LVIDd of control mice except for mice treated with AC that showed mild reduction in LVIDd compare to control (FIGS. 4C and 4D). Those results Indicates that injecting AC or Sphk1 to the LV during acute MI results in better heart function in treated mice compared to the control.

In accordance with the beneficial effect that AC and Sphk1 have on heart function a significant reduction in the scar size 29 days post MI was found. In mice treated with AC, Sphk1 or AC +Sphk1 the scar areas were 14.2%, 16.7% and 16.1% of LV area compared to 23.3% in control mice (FIG. 4D and FIG. 9C).

Overall, these data identify AC as an important component of the in vivo/in vitro oocyte and embryo environment, and provide a novel technology for enhancing the outcome of assisted fertilization.

Table 1 contains the nucleotide sequences to be encoded by the modRNAs of the present method.

TABLE 1

| Gene | Open Reading Frame |
|---|---|
| ASAH1 | ATGCCGGGCCGGAGTTGCGTCGCCTTAGTCCTCCTGGCTGCCGCCGTCAGCTGTGCCGTCGCGCA |
| transcript | GCACGCGCCGCCGTGGACAGAGGACTGCAGAAAATCAACCTATCCTCCTTCAGGACCAACGTACA |
| variant 1 | GAGGTGCAGTTCCATGGTACACCATAAATCTTGACTTACCACCCTACAAAAGATGGCATGAATTG |
| (ACv1) | ATGCTTGACAAGGCACCAGTGCTAAAGGTTATAGTGAATTCTCTGAAGAATATGATAAATACATT |
| | CGTGCCAAGTGGAAAAATTATGCAGGTGGTGGATGAAAAATTGCCTGGCCTACTTGGCAACTTTC |
| | CTGGCCCTTTTGAAGAGGAAATGAAGGGTATTGCCGCTGTTACTGATATACCTTTAGGAGAGATT |
| | ATTTCATTCAATATTTTTTATGAATTATTTACCATTTGTACTTCAATAGTAGCAGAAGACAAAAA |
| | AGGTCATCTAATACATGGGAGAAACATGGATTTTGGAGTATTTCTTGGGTGGAACATAAATAATG |
| | ATACCTGGGTCATAACTGAGCAACTAAAACCTTTAACAGTGAATTTGGATTTCCAAAGAAACAAC |
| | AAAACTGTCTTCAAGGCTTCAAGCTTTGCTGGCTATGTGGGCATGTTAACAGGATTCAAACCAGG |
| | ACTGTTCAGTCTTACACTGAATGAACGTTTCAGTATAAATGGTGGTTATCTGGGTATTCTAGAAT |
| | GGATTCTGGGAAAGAAAGATGTCATGTGGATAGGGTTCCTCACTAGAACAGTTCTGGAAAATAGC |
| | ACAAGTTATGAAGAAGCCAAGAATTTATTGACCAAGACCAAGATATTGGCCCCAGCCTACTTTAT |
| | CCTGGGAGGCAACCAGTCTGGGGAAGGTTGTGTGATTACACGAGACAGAAAGGAATCATTGGATG |
| | TATATGAACTCGATGCTAAGCAGGGTAGATGGTATGTGGTACAAACAAATTATGACCGTTGGAAA |
| | CATCCCTTCTTCCTTGATGATCGCAGAACGCCTGCAAAGATGTGTCTGAACCGCACCAGCCAAGA |
| | GAATATCTCATTTGAAACCATGTATGATGTCCTGTCAACAAAACCTGTCCTCAACAAGCTGACCG |
| | TATACACAACCTTGATAGATGTTACCAAAGGTCAATTCGAAACTTACCTGCGGGACTGCCCTGAC |
| | CCTTGTATAGGTTGGTGA (SEQ ID NO: 1) |
| Sphk1 | ATGGATCCAGTGGTCGGTTGCGGACGTGGCCTCTTTGGTTTTGTTTTCTCAGCGGGCGGCCCCCG |
| | GGGCGTGCTCCCGCGGCCCTGCCGCGTGCTGGTGCTGCTGAACCCGCGCGGCGGCAAGGGCAAGG |
| | CCTTGCAGCTCTTCCGGAGTCACGTGCAGCCCCTTTTGGCTGAGGCTGAAATCTCCTTCACGCTG |
| | ATGCTCACTGAGCGGCGGAACCACGCGCGGGAGCTGGTGCGGTCGGAGGAGCTGGGCCGCTGGGA |
| | CGCTCTGGTGGTCATGTCTGGAGACGGGCTGATGCACGAGGTGGTGAACGGGCTCATGGAGCGGC |
| | CTGACTGGGAGACCGCCATCCAGAAGCCCCTGTGTAGCCTCCCAGCAGGCTCTGGCAACGCGCTG |
| | GCAGCTTCCTTGAACCATTATGCTGGCTATGAGCAGGTCACCAATGAAGACCTCCTGACCAACTG |
| | CACGCTATTGCTGTGCCGCCGGCTGCTGTCACCCATGAACCTGCTGTCTCTGCACACGGCTTCGG |
| | GGCTGCGCCTCTTCTCTGTGCTCAGCCTGGCCTGGGGCTTCATTGCTGATGTGGACCTAGAGAGT |
| | GAGAAGTATCGGCGTCTGGGGGAGATGCGCTTCACTCTGGGCACCTTCCTGCGTCTGGCAGCCCT |
| | GCGCACCTACCGCGGCCGACTGGCCTACCTCCCTGTAGGAAGAGTGGGTTCCAAGACACCTGCCT |
| | CCCCCGTTGTGGTCCAGCAGGGCCCGGTAGATGCACACCTTGTGCCACTGGAGGAGCCAGTGCCC |
| | TCTCACTGGACAGTGGTGCCCGACGAGGACTTTGTGCTAGTCCTGGCACTGCTGCACTCGCACCT |
| | GGGCAGTGAGATGTTTGCTGCACCCATGGGCCGCTGTGCAGCTGGCGTCATGCATCTGTTCTACG |
| | TGCGGGCGGGAGTGTCTCGTGCCATGCTGCTGCGCCTCTTCCTGGCCATGGAGAAGGGCAGGCAT |
| | ATGGAGTATGAATGCCCCTACTTGGTATATGTGCCCGTGGTCGCCTTCCGCTTGGAGCCCAAGGA |
| | TGGGAAAGGTGTGTTTGCAGTGGATGGGGAATTGATGGTTAGCGAGGCCGTGCAGGGCCAGGTGC |
| | ACCCAAACTACTTCTGGATGGTCAGCGGTTGCGTGGAGCCCCCGCCCAGCTGGAAGCCCCAGCAG |
| | ATGCCACCGCCAGAAGAGCCCTTATGA (SEQ ID NO: 2) |
| S1PR2 | ATGGGCAGCTTGTACTCGGAGTACCTGAACCCCAACAAGGTCCAGGAACACTATAATTATACCAA |
| | GGAGACGCTGGAAACGCAGGAGACGACCTCCCGCCAGGTGGCCTCGGCCTTCATCGTCATCCTCT |
| | GTTGCGCCATTGTGGTGGAAAACCTTCTGGTGCTCATTGCGGTGGCCCGAAACAGCAAGTTCCAC |
| | TCGGCAATGTACCTGTTTCTGGGCAACCTGGCCGCCTCCGATCTACTGGCAGGCGTGGCCTTCGT |
| | AGCCAATACCTTGCTCTCTGGCTCTGTCACGCTGAGGCTGACGCCTGTGCAGTGGTTTGCCCGGG |
| | AGGGCTCTGCCTTCATCACGCTCTCGGCCTCTGTCTTCAGCCTCCTGGCCATCGCCATTGAGCGC |
| | CACGTGGCCATTGCCAAGGTCAAGCTGTATGGCAGCGACAAGAGCTGCCGCATGCTTCTGCTCAT |
| | CGGGGCCTCGTGGCTCATCTCGCTGGTCCTCGGTGGCCTGCCCATCCTTGGCTGGAACTGCCTGG |
| | GCCACCTCGAGGCCTGCTCCACTGTCCTGCCTCTCTACGCCAAGCATTATGTGCTGTGCGTGGTG |
| | ACCATCTTCTCCATCATCCTGTTGGCCATCGTGGCCCTGTACGTGCGCATCTACTGCGTGGTCCG |
| | CTCAAGCCACGCTGACATGGCCGCCCCGCAGACGCTAGCCCTGCTCAAGACGGTCACCATCGTGC |
| | TAGGCGTCTTTATCGTCTGCTGGCTGCCCGCCTTCAGCATCCTCCTTCTGGACTATGCCTGTCCC |
| | GTCCACTCCTGCCCGATCCTCTACAAAGCCCACTACTTTTTCGCCGTCTCCACCCTGAATTCCCT |
| | GCTCAACCCCGTCATCTACACGTGGCGCAGCCGGGACCTGCGGCGGGAGGTGCTTCGGCCGCTGC |
| | AGTGCTGGAGGCCGGGGGTGGGGGTGCAAGGACGGAGGCGGGGCGGGACCCCGGGCCACCACCTC |
| | CTGCCACTCCGCAGCTCCAGCTCCCTGGAGAGGGGCATGCACATGCCCACGTCACCCACGTTTCT |
| | GGAGGGCAACACGGTGGTCATG (SEQ ID NO: 3) |
| Firefly | ATGGCCGATGCTAAGAACATTAAGAAGGGCCCTGCTCCCTTCTACCCTCTGGAGGATGGCACCGC |
| luciferase | TGGCGAGCAGCTGCACAAGGCCATGAAGAGGTATGCCCTGGTGCCTGGCACCATTGCCTTCACCG |
| | ATGCCCACATTGAGGTGGACATCACCTATGCCGAGTACTTCGAGATGTCTGTGCGCCTGGCCGAG |
| | GCCATGAAGAGGTACGGCCTGAACACCAACCACCGCATCGTGGTGTGCTCTGAGAACTCTCTGCA |
| | GTTCTTCATGCCAGTGCTGGGCGCCCTGTTCATCGGAGTGGCCGTGGCCCCTGCTAACGACATTT |
| | ACAACGAGCGCGAGCTGCTGAACAGCATGGGCATTTCTCAGCCTACCGTGGTGTTCGTGTCTAAG |
| | AAGGGCCTGCAGAAGATCCTGAACGTGCAGAAGAAGCTGCCTATCATCCAGAAGATCATCATCAT |
| | GGACTCTAAGACCGACTACCAGGGCTTCCAGAGCATGTACACATTCGTGACATCTCATCTGCCTC |
| | CTGGCTTCAACGAGTACGACTTCGTGCCAGAGTCTTTCGACAGGGACAAAACCATTGCCCTGATC |
| | ATGAACAGCTCTGGGTCTACCGGCCTGCCTAAGGGCGTGGCCCTGCCTCATCGCACCGCCTGTGT |
| | GCGCTTCTCTCACGCCCGCGACCCTATTTTCGGCAACCAGATCATCCCCGACACCGCTATTCTGA |
| | GCGTGGTGCCATTCCACCACGGCTTCGGCATGTTCACCACCCTGGGCTACCTGATTTGCGGCTTT |
| | CGGGTGGTGCTGATGTACCGCTTCGAGGAGGAGCTGTTCCTGCGCAGCCTGCAAGACTACAAAAT |
| | TCAGTCTGCCCTGCTGGTGCCAACCCTGTTCAGCTTCTTCGCTAAGAGCACCCTGATCGACAAGT |
| | ACGACCTGTCTAACCTGCACGAGATTGCCTCTGGCGGCGCCCCACTGTCTAAGGAGGTGGGCGAA |
| | GCCGTGGCCAAGCGCTTTCATCTGCCAGGCATCCGCCAGGGCTACGGCCTGACCGAGACAACCAG |
| | CGCCATTCTGATTACCCCAGAGGGCGACGACAAGCCTGGCGCCGTGGGCAAGGTGGTGCCATTCT |
| | TCGAGGCCAAGGTGGTGGACCTGGACACCGGCAAGACCCTGGGAGTGAACCAGCGCGGCGAGCTG |
| | TGTGTGCGCGGCCCTATGATTATGTCCGGCTACGTGAATAACCCTGAGGCCACAAACGCCCTGAT |
| | CGACAAGGACGGCTGGCTGCACTCTGGCGACATTGCCTACTGGGACGAGGACGAGCACTTCTTCA |
| | TCGTGGACCGCCTGAAGTCTCTGATCAAGTACAAGGGCTACCAGGTGGCCCCAGCCGAGCTGGAG |
| | TCTATCCTGCTGCAGCACCCTAACATTTTCGACGCCGGAGTGGCCGGCCTGCCCGACGACGATGC |
| | CGGCGAGCTGCCTGCCGCCGTCGTCGTGCTGGAACACGGCAAGACCATGACCGAGAAGGAGATCG |
| | TGGACTATGTGGCCAGCCAGGTGACAACCGCCAAGAAGCTGCGCGGCGGAGTGGTGTTCGTGGAC |
| | GAGGTGCCCAAGGGCCTGACCGGCAAGCTGGACGCCCGCAAGATCCGCGAGATCCTGATCAAGGC |
| | TAAGAAAGGCGGCAAGATCGCCGTGTAA (SEQ ID NO: 4) |
| nGFP | ATGGTGAGCAAGGGCGAGGAGCTGTTCACCGGGGTGGTGCCCATCCTGGTCGAGCTGGACGGCGA |
| | CGTAAACGGCCACAAGTTCAGCGTGTCCGGCGAGGGCGAGGGCGATGCCACCTACGGCAAGCTGA |
| | CCCTGAAGTTCATCTGCACCACCGGCAAGCTGCCCGTGCCCTGGCCCACCCTCGTGACCACCCTG |
| | ACCTACGGCGTGCAGTGCTTCAGCCGCTACCCCGACCACATGAAGCAGCACGACTTCTTCAAGTC |
| | CGCCATGCCCGAAGGCTACGTCCAGGAGCGCACCATCTTCTTCAAGGACGACGGCAACTACAAGA |
| | CCCGCGCCGAGGTGAAGTTCGAGGGCGACACCCTGGTGAACCGCATCGAGCTGAAGGGCATCGAC |
| | TTCAAGGAGGACGGCAACATCCTGGGGCACAAGCTGGAGTACAACTACAACAGCCACAACGTCTA |
| | TATCATGGCCGACAAGCAGAAGAACGGCATCAAGGTGAACTTCAAGATCCGCCACAACATCGAGG |
| | ACGGCAGCGTGCAGCTCGCCGACCACTACCAGCAGAACACCCCCATCGGCGACGGCCCCGTGCTG |
| | CTGCCCGACAACCACTACCTGAGCACCCAGTCCGCCCTGAGCAAAGACCCCAACGAGAAGCGCGA |
| | TCACATGGTCCTGCTGGAGTTCGTGACCGCCGCCGGGATCACTCTCGGCATGGACGAGCTGTACA |
| | AGGGAGATCCAAAAAAGAAGAGAAAGGTAGGCGATCCAAAAAAGAAGAGAAAGGTAGGTGATCCA |
| | AAAAAGAAGAGAAAGGTATAA (SEQ ID NO: 5) |
| ASAH2 | ATGAACTGCTGCATCGGGCTGGGAGAGAAAGCTCGCGGGTCCCACCGGGCCTCCTACCCAAGTCT |
| transcript | CAGCGCGCTTTTCACCGAGGCCTCAATTCTGGGATTTGGCAGCTTTGCTGTGAAAGCCCAATGGA |
| variant 2 | CAGAGGACTGCAGAAAATCAACCTATCCTCCTTCAGGACCAACGTACAGAGGTGCAGTTCCATGG |
| (ACv2) | TACACCATAAATCTTGACTTACCACCCTACAAAAGATGGCATGAATTGATGCTTGACAAGGCACC |
| | AGTGCTAAAGGTTATAGTGAATTCTCTGAAGAATATGATAAATACATTCGTGCCAAGTGGAAAAA |
| | TTATGCAGGTGGTGGATGAAAAATTGCCTGGCCTACTTGGCAACTTTCCTGGCCCTTTTGAAGAG |
| | GAAATGAAGGGTATTGCCGCTGTTACTGATATACCTTTAGGAGAGATTATTTCATTCAATATTTT |
| | TTATGAATTATTTACCATTTGTACTTCAATAGTAGCAGAAGACAAAAAAGGTCATCTAATACATG |
| | GGAGAAACATGGATTTTGGAGTATTTCTTGGGTGGAACATAAATAATGATACCTGGGTCATAACT |
| | GAGCAACTAAAACCTTTAACAGTGAATTTGGATTTCCAAAGAAACAACAAAACTGTCTTCAAGGC |
| | TTCAAGCTTTGCTGGCTATGTGGGCATGTTAACAGGATTCAAACCAGGACTGTTCAGTCTTACAC |
| | TGAATGAACGTTTCAGTATAAATGGTGGTTATCTGGGTATTCTAGAATGGATTCTGGGAAAGAAA |
| | GATGTCATGTGGATAGGGTTCCTCACTAGAACAGTTCTGGAAAATAGCACAAGTTATGAAGAAGC |
| | CAAGAATTTATTGACCAAGACCAAGATATTGGCCCCAGCCTACTTTATCCTGGGAGGCAACCAGT |
| | CTGGGGAAGGTTGTGTGATTACACGAGACAGAAAGGAATCATTGGATGTATATGAACTCGATGCT |
| | AAGCAGGGTAGATGGTATGTGGTACAAACAAATTATGACCGTTGGAAACATCCCTTCTTCCTTGA |
| | TGATCGCAGAACGCCTGCAAAGATGTGTCTGAACCGCACCAGCCAAGAGAATATCTCATTTGAAA |
| | CCATGTATGATGTCCTGTCAACAAAACCTGTCCTCAACAAGCTGACCGTATACACAACCTTGATA |
| | GATGTTACCAAAGGTCAATTCGAAACTTACCTGCGGGACTGCCCTGACCCTTGTATAGGTTGGTG |
| | A (SEQ ID NO: 6) |
| ASAH1 | ATGAACTGCTGCATCGGGCTGGGAGAGAAAGCTCGCGGGTCCCACCGGGCCTCCTACCCAAGTCT |
| transcript | CAGCGCGCTTTTCACCGAGGCCTCAATTCTGGGATTTGGCAGCTTTGCTGTGAAAGCCCAATGGA |
| variant 3 | CAGAGGACTGCAGAAAATCAACCTATCCTCCTTCAGGACCAACTGTCTTCCCTGCTGTTATAAGG |
| | TACAGAGGTGCAGTTCCATGGTACACCATAAATCTTGACTTACCACCCTACAAAAGATGGCATGA |
| | ATTGATGCTTGACAAGGCACCAGTGCCTGGCCTACTTGGCAACTTTCCTGGCCCTTTTGAAGAGG |
| | AAATGAAGGGTATTGCCGCTGTTACTGATATACCTTTAGGAGAGATTATTTCATTCAATATTTTT |
| | TATGAATTATTTACCATTTGTACTTCAATAGTAGCAGAAGACAAAAAAGGTCATCTAATACATGG |
| | GAGAAACATGGATTTTGGAGTATTTCTTGGGTGGAACATAAATAATGATACCTGGGTCATAACTG |
| | AGCAACTAAAACCTTTAACAGTGAATTTGGATTTCCAAAGAAACAACAAAACTGTCTTCAAGGCT |
| | TCAAGCTTTGCTGGCTATGTGGGCATGTTAACAGGATTCAAACCAGGACTGTTCAGTCTTACACT |
| | GAATGAACGTTTCAGTATAAATGGTGGTTATCTGGGTATTCTAGAATGGATTCTGGGAAAGAAAG |
| | ATGTCATGTGGATAGGGTTCCTCACTAGAACAGTTCTGGAAAATAGCACAAGTTATGAAGAAGCC |
| | AAGAATTTATTGACCAAGACCAAGATATTGGCCCCAGCCTACTTTATCCTGGGAGGCAACCAGTC |
| | TGGGGAAGGTTGTGTGATTACACGAGACAGAAAGGAATCATTGGATGTATATGAACTCGATGCTA |
| | AGCAGGGTAGATGGTATGTGGTACAAACAAATTATGACCGTTGGAAACATCCCTTCTTCCTTGAT |
| | GATCGCAGAACGCCTGCAAAGATGTGTCTGAACCGCACCAGCCAAGAGAATATCTCATTTGAAAC |
| | CATGTATGATGTCCTGTCAACAAAACCTGTCCTCAACAAGCTGACCGTATACACAACCTTGATAG |
| | ATGTTACCAAAGGTCAATTCGAAACTTACCTGCGGGACTGCCCTGACCCTTGTATAGGTTGGTGA |
| | (SEQ ID NO: 7) |
| ASAH2 | [symbol] GCCAAACGCACCTTCTCTAACTTGGAGACATTCCTGATTTTCCTCCTTGTAATGATGAGTGC |
| transcript | CATCACAGTGGCCCTTCTCAGCCTCTTGTTTATCACCAGTGGGACCATTGAAAACCACAAAGATT |
| variant 1 | TAGGAGGCCATTTTTTTTCAACCACCCAAAGCCCTCCAGCCACCCAGGGCTCCACAGCTGCCCAA |
| | CGCTCCACAGCCACCCAGCATTCCACAGCCACCCAGAGCTCCACAGCCACTCAAACTTCTCCAGT |
| | GCCTTTAACCCCAGAGTCTCCTCTATTTCAGAACTTCAGTGGCTACCATATTGGTGTTGGACGAG |
| | CTGACTGCACAGGACAAGTAGCAGATATCAATTTGATGGGCTATGGCAAATCCGGCCAGAATGCA |
| | CAGGGCATCCTCACCAGGCTATACAGTCGTGCCTTCATCATGGCAGAACCTGATGGGTCCAATCG |
| | AACAGTGTTTGTCAGCATCGACATAGGCATGGTATCACAAAGGCTCAGGCTGGAGGTCCTGAACA |
| | GACTGCAGAGTAAATATGGCTCCCTGTACAGAAGAGATAATGTCATCCTGAGTGGCACTCACACT |
| | CATTCAGGTCCTGCAGGATATTTCCAGTATACCGTGTTTGTAATTGCCAGTGAAGGATTTAGCAA |
| | TCAAACTTTTCAGCACATGGTCACTGGTATCTTGAAGAGCATTGACATAGCACACACAAATATGA |
| | AACCAGGCAAAATCTTCATCAATAAAGGAAATGTGGATGGTGTGCAGATCAACAGAAGTCCGTAT |
| | TCTTACCTTCAAAATCCGCAGTCAGAGAGAGCAAGGTATTCTTCAAATACAGACAAGGAAATGAT |
| | AGTTTTGAAAATGGTAGATTTGAATGGAGATGACTTGGGCCTTATCAGCTGGTTTGCCATCCACC |
| | CGGTCAGCATGAACAACAGTAACCATCTTGTAAACAGTGACAATGTGGGCTATGCATCTTACCTG |
| | CTTGAGCAAGAGAAGAACAAAGGATATCTACCTGGACAGGGGCCATTTGTAGCAGCCTTTGCTTC |
| | ATCAAACCTAGGAGATGTGTCCCCCAACATTCTTGGACCACGTTGCATCAACACAGGAGAGTCCT |
| | GTGATAACGCCAATAGCACTTGTCCCATTGGTGGGCCTAGCATGTGCATTGCTAAGGGACCTGGA |
| | CAGGATATGTTTGACAGCACACAAATTATAGGACGGGCCATGTATCAGAGAGCAAAGGAACTCTA |
| | TGCCTCTGCCTCCCAGGAGGTAACAGGACCACTGGCTTCAGCACACCAGTGGGTGGATATGACAG |
| | ATGTGACTGTCTGGCTCAATTCCACACATGCATCAAAAACATGTAAACCAGCATTGGGCTACAGT |
| | TTTGCAGCTGGCACTATTGATGGAGTTGGAGGCCTCAATTTTACACAGGGGAAAACAGAAGGGGA |
| | TCCATTTTGGGACACCATTCGGGACCAGATCCTGGGAAAGCCATCTGAAGAAATTAAAGAATGTC |
| | ATAAACCAAAGCCCATCCTTCTTCACACCGGAGAACTATCAAAACCTCACCCCTGGCATCCAGAC |
| | ATTGTTGATGTTCAGATTATTACCCTTGGGTCCTTGGCCATAACTGCCATCCCCGGGGAGTTTAC |
| | GACCATGTCTGGACGAAGACTTCGAGAGGCAGTTCAAGCAGAATTTGCATCTCATGGGATGCAGA |
| | ACATGACTGTTGTTATTTCAGGTCTATGCAACGTCTATACACATTACATTACCACTTATGAAGAA |
| | TACCAGGCTCAGCGATATGAGGCAGCATCGACAATTTATGGACCGCACACATTATCTGCTTACAT |
| | TCAGCTCTTCAGAAACCTTGCTAAGGCTATTGCTACGGACACGGTAGCCAACCTGAGCAGAGGTC |
| | CAGAACCTCCCTTTTTCAAACAATTAATAGTTCCATTAATTCCTAGTATTGTGGATAGAGCACCA |
| | AAAGGCAGAACTTTCGGGGATGTCCTGCAGCCAGCAAAACCTGAATACAGAGTGGGGGAAGTTGC |
| | TGAAGTTATATTTGTAGGTGCTAACCCGAAGAATTCAGTACAAAACCAGACCCATCAGACCTTCC |
| | TCACTGTGGAGAAATATGAGGCTACTTCAACATCGTGGCAGATAGTGTGTAATGATGCCTCCTGG |
| | GAGACTCGTTTTTATTGGCACAAGGGACTCCTGGGTCTGAGTAATGCAACAGTGGAATGGCATAT |
| | TCCAGACACTGCCCAGCCTGGAATCTACAGAATAAGATATTTTGGACACAATCGGAAGCAGGACA |
| | TTCTGAAGCCTGCTGTCATACTTTCATTTGAAGGCACTTCCCCGGCTTTTGAAGTTGTAACTATT |
| | TAG [symbol] (SEQ ID NO: 8) |
| ASAH2 | [symbol] GCCAAACGCACCTTCTCTAACTTGGAGACATTCCTGATTTTCCTCCTTGTAATGATGAGTGC |
| transcript | CATCACAGTGGCCCTTCTCAGCCTCTTGTTTATCACCAGTGGGACCATTGAAAACCACAAAGATT |
| variant 2 | TAGGAGGCCATTTTTTTTCAACCACCCAAAGCCCTCCAGCCACCCAGGGCTCCACAGCTGCCCAA |
| | CGCTCCACAGCCACCCAGCATTCCACAGCCACCCAGAGCTCCACAGCCACTCAAACTTCTCCAGT |
| | GCCTTTAACCCCAGAGTCTCCTCTATTTCAGAACTTCAGTGGCTACCATATTGGTGTTGGACGAG |
| | CTGACTGCACAGGACAAGTAGCAGATATCAATTTGATGGGCTATGGCAAATCCGGCCAGAATGCA |
| | CAGGGCATCCTCACCAGGCTATACAGTCGTGCCTTCATCATGGCAGAACCTGATGGGTCCAATCG |
| | AACAGTGTTTGTCAGCATCGACATAGGCATGGTATCACAAAGGCTCAGGCTGGAGGTCCTGAACA |
| | GACTGCAGAGTAAATATGGCTCCCTGTACAGAAGAGATAATGTCATCCTGAGTGGCACTCACACT |
| | CATTCAGGTCCTGCAGGATATTTCCAGTATACCGTGTTTGTAATTGCCAGTGAAGGATTTAGCAA |
| | TCAAACTTTTCAGCACATGGTCACTGGTATCTTGAAGAGCATTGACATAGCACACACAAATATGA |
| | AACCAGGCAAAATCTTCATCAATAAAGGAAATGTGGATGGTGTGCAGATCAACAGAAGTCCGTAT |
| | TCTTACCTTCAAAATCCGCAGTCAGAGAGAGCAAGGTATTCTTCAAATACAGACAAGGAAATGAT |
| | AGTTTTGAAAATGGTAGATTTGAATGGAGATGACTTGGGCCTTATCAGCTGGTTTGCCATCCACC |
| | CGGTCAGCATGAACAACAGTAACCATCTTGTAAACAGTGACAATGTGGGCTATGCATCTTACCTG |
| | CTTGAGCAAGAGAAGAACAAAGGATATCTACCTGGACAGGGGCCATTTGTAGCAGCCTTTGCTTC |
| | ATCAAACCTAGGAGATGTGTCCCCCAACATTCTTGGACCACGTTGCATCAACACAGGAGAGTCCT |
| | GTGATAACGCCAATAGCACTTGTCCCATTGGTGGGCCTAGCATGTGCATTGCTAAGGGACCTGGA |
| | CAGGATATGTTTGACAGCACACAAATTATAGGACGGGCCATGTATCAGAGAGCAAAGTCAAAAAC |
| | ATGTAAACCAGCATTGGGCTACAGTTTTGCAGCTGGCACTATTGATGGAGTTGGAGGCCTCAATT |
| | TTACACAGGGGAAAACAGAAGGGGATCCATTTTGGGACACCATTCGGGACCAGATCCTGGGAAAG |
| | CCATCTGAAGAAATTAAAGAATGTCATAAACCAAAGCCCATCCTTCTTCACACCGGAGAACTATC |
| | AAAACCTCACCCCTGGCATCCAGACATTGTTGATGTTCAGATTATTACCCTTGGGTCCTTGGCCA |
| | TAACTGCCATCCCCGGGGAGTTTACGACCATGTCTGGACGAAGACTTCGAGAGGCAGTTCAAGCA |
| | GAATTTGCATCTCATGGGATGCAGAACATGACTGTTGTTATTTCAGGTCTATGCAACGTCTATAC |
| | ACATTACATTACCACTTATGAAGAATACCAGGCTCAGCGATATGAGGCAGCATCGACAATTTATG |
| | GACCGCACACATTATCTGCTTACATTCAGCTCTTCAGAAACCTTGCTAAGGCTATTGCTACGGAC |
| | ACGGTAGCCAACCTGAGCAGAGGTCCAGAACCTCCCTTTTTCAAACAATTAATAGTTCCATTAAT |
| | TCCTAGTATTGTGGATAGAGCACCAAAAGGCAGAACTTTCGGGGATGTCCTGCAGCCAGCAAAAC |
| | CTGAATACAGAGTGGGGGAAGTTGCTGAAGTTATATTTGTAGGTGCTAACCCGAAGAATTCAGTA |
| | CAAAACCAGACCCATCAGACCTTCCTCACTGTGGAGAAATATGAGGCTACTTCAACATCGTGGCA |
| | GATAGTGTGTAATGATGCCTCCTGGGAGACTCGTTTTTATTGGCACAAGGGACTCCTGGGTCTGA |
| | GTAATGCAACAGTGGAATGGCATATTCCAGACACTGCCCAGCCTGGAATCTACAGAATAAGATAT |
| | TTTGGACACAATCGGAAGCAGGACATTCTGAAGCCTGCTGTCATACTTTCATTTGAAGGCACTTC |
| | CCCGGCTTTTGAAGTTGTAACTATTTAGT [symbol] (SEQ ID NO: 9) |
| ASAH2B | [symbol] AGGCAGCATCGACAATTTATGGACCGCACGCATTATCTGCTTACATTCAGCTCTTCAGAAAC |
| transcript | CTTGCTAAGGCTATTGCTACGTATTGTGGATAGAGCACCAAAAGGCAGAACTTTCGGGGATGTCC |
| variant 1 | TGCAGCCAGCAAAACCTGAATACAGAGTGGGGGAAGTTGCTGAAGTTATATTTGTAGGTGCTAAC |
| | CCGAAGAATTCAGTACAAAACCAGACCCATCAGACCTTCCTCACTGTGGAGAAATATGAGGCTAC |
| | TTCAACATCGTGGCAGATAGTGTGTAATGATGCCTCCTGGGAGACTCGTTTTTATTGGCACAAGG |
| | GACTCCTGGGTCTGAGTAATGCAACAGTGGAATGGCATATTCCAGACACTGCCCAGCCTGGAATC |
| | TACAGAATAAGATATTTTGGACACAATCGGAAGCAGGACATTCTGAAGCCTGCTGTCATACTTTC |
| | ATTTGAAGGCACTTCCCCGGCTTTTGAAGTTGTAACTATTTAG [symbol] (SEQ ID NO: 10) |
| ASAH2B | [symbol] GTAGCCAACCTGAGCAGAGGTCCAGAACCTCCCTTTTTCAAACAATTAATAGTTCCATTAAT |
| transcript | TCCTAGTATTGTGGATAGAGCACCAAAAGGCAGAACTTTCGGGGATGTCCTGCAGCCAGCAAAAC |
| variant 3 | CTGAATACAGAGTGGGGGAAGTTGCTGAAGTTATATTTGTAGGTGCTAACCCGAAGAATTCAGTA |
| | CAAAACCAGACCCATCAGACCTTCCTCACTGTGGAGAAATATGAGGCTACTTCAACATCGTGGCA |
| | GATAGTGTGTAATGATGCCTCCTGGGAGACTCGTTTTTATTGGCACAAGGGACTCCTGGGTCTGA |
| | GTAATGCAACAGTGGAATGGCATATTCCAGACACTGCCCAGCCTGGAATCTACAGAATAAGATAT |
| | TTTGGACACAATCGGAAGCAGGACATTCTGAAGCCTGCTGTCATACTTTCATTTGAAGGCACTTC |
| | CCCGGCTTTTGAAGTTGTAACTATTTAGTGAATGGTAGCCAACCTGAGCAGAGGTCCAGAACCTC |
| | CCTTTTTCAAACAATTAATAGTTCCATTAATTCCTAGTATTGTGGATAGAGCACCAAAAGGCAGA |
| | ACTTTCGGGGATGTCCTGCAGCCAGCAAAACCTGAATACAGAGTGGGGGAAGTTGCTGAAGTTAT |
| | ATTTGTAGGTGCTAACCCGAAGAATTCAGTACAAAACCAGACCCATCAGACCTTCCTCACTGTGG |
| | AGAAATATGAGGCTACTTCAACATCGTGGCAGATAGTGTGTAATGATGCCTCCTGGGAGACTCGT |
| | TTTTATTGGCACAAGGGACTCCTGGGTCTGAGTAATGCAACAGTGGAATGGCATATTCCAGACAC |
| | TGCCCAGCCTGGAATCTACAGAATAAGATATTTTGGACACAATCGGAAGCAGGACATTCTGAAGC |
| | CTGCTGTCATACTTTCATTTGAAGGCACTTCCCCGGCTTTTGAAGTTGTAACTATTTAG [symbol] |
| | (SEQ ID NO: 11) |
| ASAH2B | [symbol] GTAGCCAACCTGAGCAGAGGTCCAGAACCTCCCTTTTTCAAACAATTAATAGTTCCATTAAT |
| transcript | TCCTAGTATTGTGGATAGAGCACCAAAAGGCAGAACTTTCGGGGATGTCCTGCAGCCAGCAAAAC |
| variant 4 | CTGAATACAGAGTGGGGGAAGTTGCTGAAGTTATATTTGTAGGTGCTAACCCGAAGAATTCAGTA |
| | CAAAACCAGACCCATCAGACCTTCCTCACTGTGGAGAAATATGAGGCTACTTCAACATCGTGGCA |
| | GATAGTGTGTAATGATGCCTCCTGGGAGACTCGTTTTTATTGGCACAAGGGACTCCTGGGTCTGA |
| | GTAATGCAACAGTGGAATGGCATATTCCAGACACTGCCCAGCCTGGAATCTACAGAATAAGATAT |
| | TTTGGACACAATCGGAAGCAGGACATTCTGAAGCCTGCTGTCATACTTTCATTTGAAGGCACTTC |
| | CCCGGCTTTTGAAGTTGTAACTATT [symbol] (SEQ ID NO: 12) |
| ACER1 | [symbol] CCTAGCATCTTCGCCTATCAGAGCTCCGAGGTGGACTGGTGTGAGAGCAACTTCCAGTACTC |
| | GGAGCTGGTGGCCGAGTTCTACAACACGTTCTCCAATATCCCCTTCTTCATCTTCGGGCCACTGA |
| | TGATGCTCCTGATGCACCCGTATGCCCAGAAGCGCTCCCGCTACATTTACGTTGTCTGGGTCCTC |
| | TTCATGATCATAGGCCTGTTCTCCATGTATTTCCACATGACGCTCAGCTTCCTGGGCCAGCTGCT |
| | GGACGAGATCGCCATCCTGTGGCTCCTGGGCAGTGGCTATAGCATATGGATGCCCCGCTGCTATT |
| | TCCCCTCCTTCCTTGGGGGGAACAGGTCCCAGTTCATCCGCCTGGTCTTCATCACCACTGTGGTC |
| | AGCACCCTTCTGTCCTTCCTGCGGCCCACGGTCAACGCCTACGCCCTCAACAGCATTGCCCTGCA |
| | CATTCTCTACATCGTGTGCCAGGAGTACAGGAAGACCAGCAATAAGGAGCTTCGGCACCTGATTG |
| | AGGTCTCCGTGGTTTTATGGGCTGTTGCTCTGACCAGCTGGATCAGTGACCGTCTGCTTTGCAGC |
| | TTCTGGCAGAGGATTCATTTCTTCTATCTGCACAGCATCTGGCATGTGCTCATCAGCATCACCTT |
| | CCCTTATGGCATGGTCACCATGGCCTTGGTGGATGCCAACTATGAGATGCCAGGTGAAACCCTCA |
| | AAGTCCGCTACTGGCCTCGGGACAGTTGGCCCGTGGGGCTGCCCTACGTGGAAATCCGGGGTGAT |
| | GACAAGGACTGC [symbol] (SEQ ID NO: 13) |
| ACER2 | [symbol] GGCGCCCCGCACTGGTGGGACCAGCTGCAGGCTGGTAGCTCGGAGGTGGACTGGTGCGAGGA |
| | CAACTACACCATCGTGCCTGCTATCGCCGAGTTCTACAACACGATCAGCAATGTCTTATTTTTCA |
| | TTTTACCGCCCATCTGCATGTGCTTGTTTCGTCAGTATGCAACATGCTTCAACAGTGGCATCTAC |
| | TTAATCTGGACTCTTTTGGTTGTAGTGGGAATTGGATCCGTCTACTTCCATGCAACCCTTAGTTT |
| | CTTGGGTCAGATGCTTGATGAACTTGCAGTCCTTTGGGTTCTGATGTGTGCTTTGGCCATGTGGT |
| | TCCCCAGAAGGTATCTACCAAAGATCTTTCGGAATGACCGGGGTAGGTTCAAGGTGGTGGTCAGT |
| | GTCCTGTCTGCGGTTACGACGTGCCTGGCATTTGTCAAGCCTGCCATCAACAACATCTCTCTGAT |
| | GACCCTGGGAGTTCCTTGCACTGCACTGCTCATCGCAGAGCTAAAGAGGTGTGACAACATGCGTG |
| | TGTTTAAGCTGGGCCTCTTCTCGGGCCTCTGGTGGACCCTGGCCCTGTTCTGCTGGATCAGTGAC |
| | CGAGCTTTCTGCGAGCTGCTGTCATCCTTCAACTTCCCCTACCTGCACTGCATGTGGCACATCCT |
| | CATCTGCCTTGCTGCCTACCTGGGCTGTGTATGCTTTGCCTACTTTGATGCTGCCTCAGAGATTC |
| | CTGAGCAAGGCCCTGTCATCAAGTTCTGGCCCAATGAGAAATGGGCCTTCATTGGTGTCCCCTAT |
| | GTGTCCCTCCTGTGTGCCAACAAGAAATCATCAGTCAAGATCACG [symbol] (SEQ ID NO: 14) |
| ACER3 | [symbol] GCTCCGGCCGCGGACCGAGAGGGCTACTGGGGCCCCACGACCTCCACGCTGGACTGGTGCGA |
| transcript | GGAGAACTACTCCGTGACCTGGTACATCGCCGAGTTCTGGAATACAGTGAGTAACCTGATCATGA |
| variant 1 | TTATACCTCCAATGTTCGGTGCAGTTCAGAGTGTTAGAGACGGTCTGGAAAAGCGGTACATTGCT |
| | TCTTATTTAGCACTCACAGTGGTAGGAATGGGATCCTGGTGCTTCCACATGACTCTGAAATATGA |
| | AATGCAGCTATTGGATGAACTCCCAATGATATACAGCTGTTGCATATTTGTGTACTGCATGTTTG |
| | AATGTTTCAAGATCAAGAACTCAGTAAACTACCATCTGCTTTTTACCTTAGTTCTATTCAGTTTA |
| | ATAGTAACCACAGTTTACCTTAAGGTAAAAGAGCCGATATTCCATCAGGTCATGTATGGAATGTT |
| | GGTCTTTACATTAGTACTTCGATCTATTTATATTGTTACATGGGTTTATCCATGGCTTAGAGGAC |
| | TGGGTTATACATCATTGGGTATATTTTTATTGGGATTTTTATTTTGGAATATAGATAACATATTT |
| | TGTGAGTCACTGAGGAACTTTCGAAAGAAGGTACCACCTATCATAGGTATTACCACACAATTTCA |
| | TGCATGGTGGCATATTTTAACTGGCCTTGGTTCCTATCTTCACATCCTTTTCAGTTTGTATACAA |
| | GAACACTTTACCTGAGATATAGGCCAAAAGTGAAGTTTCTCTTTGGAATCTGGCCAGTGATCCTG |
| | TTTGAGCCTCTCAGGAAGCAT [symbol] (SEQ ID NO: 15) |
| ACER3 | [symbol] GCTCCGGCCGCGGACCGAGAGGGCTACTGGGGCCCCACGACCTCCACGCTGGACTGGTGCGA |
| transcript | GGAGAACTACTCCGTGACCTGGTACATCGCCGAGTTCTTGGTAGGAATGGGATCCTGGTGCTTCC |
| variant 2 | ACATGACTCTGAAATATGAAATGCAGCTATTGGATGAACTCCCAATGATATACAGCTGTTGCATA |
| | TTTGTGTACTGCATGTTTGAATGTTTCAAGATCAAGAACTCAGTAAACTACCATCTGCTTTTTAC |
| | CTTAGTTCTATTCAGTTTAATAGTAACCACAGTTTACCTTAAGGTAAAAGAGCCGATATTCCATC |
| | AGGTCATGTATGGAATGTTGGTCTTTACATTAGTACTTCGATCTATTTATATTGTTACATGGGTT |
| | TATCCATGGCTTAGAGGACTGGGTTATACATCATTGGGTATATTTTTATTGGGATTTTTATTTTG |
| | GAATATAGATAACATATTTTGTGAGTCACTGAGGAACTTTCGAAAGAAGGTACCACCTATCATAG |
| | GTATTACCACACAATTTCATGCATGGTGGCATATTTTAACTGGCCTTGGTTCCTATCTTCACATC |
| | CTTTTCAGTTTGTATACAAGAACACTTTACCTGAGATATAGGCCAAAAGTGAAGTTTCTCTTTGG |
| | AATCTGGCCAGTGATCCTGTTTGAGCCTCTCAGGAAGCAT [symbol] (SEQ ID NO: 16) |
| ACER3 | [symbol] ATATACAGCTGTTGCATATTTGTGTACTGCATGTTTGAATGTTTCAAGATCAAGAACTCAGT |
| transcript | AAACTACCATCTGCTTTTTACCTTAGTTCTATTCAGTTTAATAGTAACCACAGTTTACCTTAAGG |
| variant 3 | TAAAAGAGCCGATATTCCATCAGGTCATGTATGGAATGTTGGTCTTTACATTAGTACTTCGATCT |
| | ATTTATATTGTTACATGGGTTTATCCATGGCTTAGAGGACTGGGTTATACATCATTGGGTATATT |
| | TTTATTGGGATTTTTATTTTGGAATATAGATAACATATTTTGTGAGTCACTGAGGAACTTTCGAA |
| | AGAAGGTACCACCTATCATAGGTATTACCACACAATTTCATGCATGGTGGCATATTTTAACTGGC |
| | CTTGGTTCCTATCTTCACATCCTTTTCAGTTTGTATACAAGAACACTTTACCTGAGATATAGGCC |
| | AAAAGTGAAGTTTCTCTTTGGAATCTGGCCAGTGATCCTGTTTGAGCCTCTCAGGAAGCAT [symbol] |
| | (SEQ ID NO: 17) |
| Sphk2 | ATGAATGGACACCTTGAAGCAGAGGAGCAGCAGGACCAGAGGCCAGACCAGGAGCTGACCGGGAG |
| | CTGGGGCCACGGGCCTAGGAGCACCCTGGTCAGGGCTAAGGCCATGGCCCCGCCCCCACCGCCAC |
| | TGGCTGCCAGCACCCCGCTCCTCCATGGCGAGTTTGGCTCCTACCCAGCCCGAGGCCCACGCTTT |
| | GCCCTCACCCTTACATCGCAGGCCCTGCACATACAGCGGCTGCGCCCCAAACCTGAAGCCAGGCC |
| | CCGGGGTGGCCTGGTCCCGTTGGCCGAGGTCTCAGGCTGCTGCACCCTGCGAAGCCGCAGCCCCT |
| | CAGACTCAGCGGCCTACTTCTGCATCTACACCTACCCTCGGGGCCGGCGCGGGGCCCGGCGCAGA |
| | GCCACTCGCACCTTCCGGGCAGATGGGGCCGCCACCTACGAAGAGAACCGTGCCGAGGCCCAGCG |
| | CTGGGCCACTGCCCTCACCTGTCTGCTCCGAGGACTGCCACTGCCCGGGGATGGGGAGATCACCC |
| | CTGACCTGCTACCTCGGCCGCCCCGGTTGCTTCTATTGGTCAATCCCTTTGGGGGTCGGGGCCTG |
| | GCCTGGCAGTGGTGTAAGAACCACGTGCTTCCCATGATCTCTGAAGCTGGGCTGTCCTTCAACCT |
| | CATCCAGACAGAACGACAGAACCACGCCCGGGAGCTGGTCCAGGGGCTGAGCCTGAGTGAGTGGG |
| | ATGGCATCGTCACGGTCTCGGGAGACGGGCTGCTCCATGAGGTGCTGAACGGGCTCCTAGATCGC |
| | CCTGACTGGGAGGAAGCTGTGAAGATGCCTGTGGGCATCCTCCCCTGCGGCTCGGGCAACGCGCT |
| | GGCCGGAGCAGTGAACCAGCACGGGGGATTTGAGCCAGCCCTGGGCCTCGACCTGTTGCTCAACT |
| | GCTCACTGTTGCTGTGCCGGGGTGGTGGCCACCCACTGGACCTGCTCTCCGTGACGCTGGCCTCG |
| | GGCTCCCGCTGTTTCTCCTTCCTGTCTGTGGCCTGGGGCTTCGTGTCAGATGTGGATATCCAGAG |
| | CGAGCGCTTCAGGGCCTTGGGCAGTGCCCGCTTCACACTGGGCACGGTGCTGGGCCTCGCCACAC |
| | TGCACACCTACCGCGGACGCCTCTCCTACCTCCCCGCCACTGTGGAACCTGCCTCGCCCACCCCT |
| | GCCCATAGCCTGCCTCGTGCCAAGTCGGAGCTGACCCTAACCCCAGACCCAGCCCCGCCCATGGC |
| | CCACTCACCCCTGCATCGTTCTGTGTCTGACCTGCCTCTTCCCCTGCCCCAGCCTGCCCTGGCCT |
| | CTCCTGGCTCGCCAGAACCCCTGCCCATCCTGTCCCTCAACGGTGGGGGCCCAGAGCTGGCTGGG |
| | GACTGGGGTGGGGCTGGGGATGCTCCGCTGTCCCCGGACCCACTGCTGTCTTCACCTCCTGGCTC |
| | TCCCAAGGCAGCTCTACACTCACCCGTCTCCGAAGGGGCCCCCGTAATTCCCCCATCCTCTGGGC |
| | TCCCACTTCCCACCCCTGATGCCCGGGTAGGGGCCTCCACCTGCGGCCCGCCCGACCACCTGCTG |
| | CCTCCGCTGGGCACCCCGCTGCCCCCAGACTGGGTGACGCTGGAGGGGGACTTTGTGCTCATGTT |
| | GGCCATCTCGCCCAGCCACCTAGGCGCTGACCTGGTGGCAGCTCCGCATGCGCGCTTCGACGACG |
| | GCCTGGTGCACCTGTGCTGGGTGCGTAGCGGCATCTCGCGGGCTGCGCTGCTGCGCCTTTTCTTG |
| | GCCATGGAGCGTGGTAGCCACTTCAGCCTGGGCTGTCCGCAGCTGGGCTACGCCGCGGCCCGTGC |
| | CTTCCGCCTAGAGCCGCTCACACCACGCGGCGTGCTCACAGTGGACGGGGAGCAGGTGGAGTATG |
| | GGCCGCTACAGGCACAGATGCACCCTGGCATCGGTACACTGCTCACTGGGCCTCCTGGCTGCCCG |
| | GGGCGGGAGCCCTGA (SEQ ID NO: 18) |

### Reducing Cell Death in Rat Myocardium

In order to characterize the dynamics of cell death as well as expression of genes that are involved in the metabolism and signaling of sphingolipids in the heart as a result of myocardial infarction (MI) in mice, hearts were infarcted by ligation of the left anterior descending artery (LAD) and harvested at different time point post ligation.

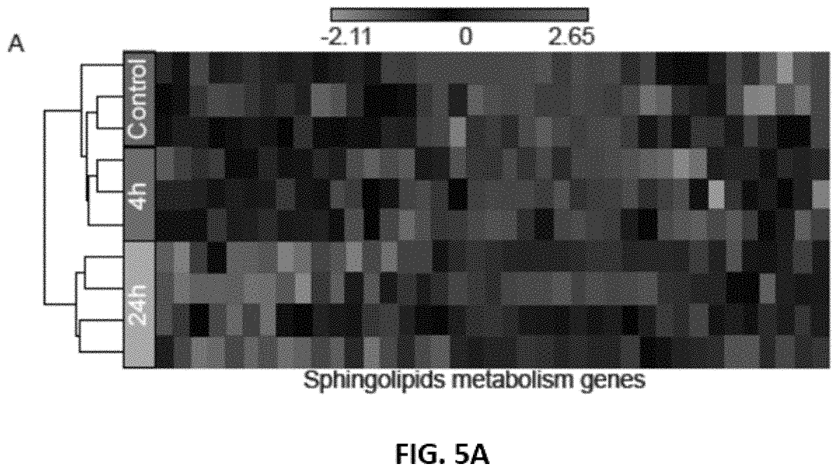

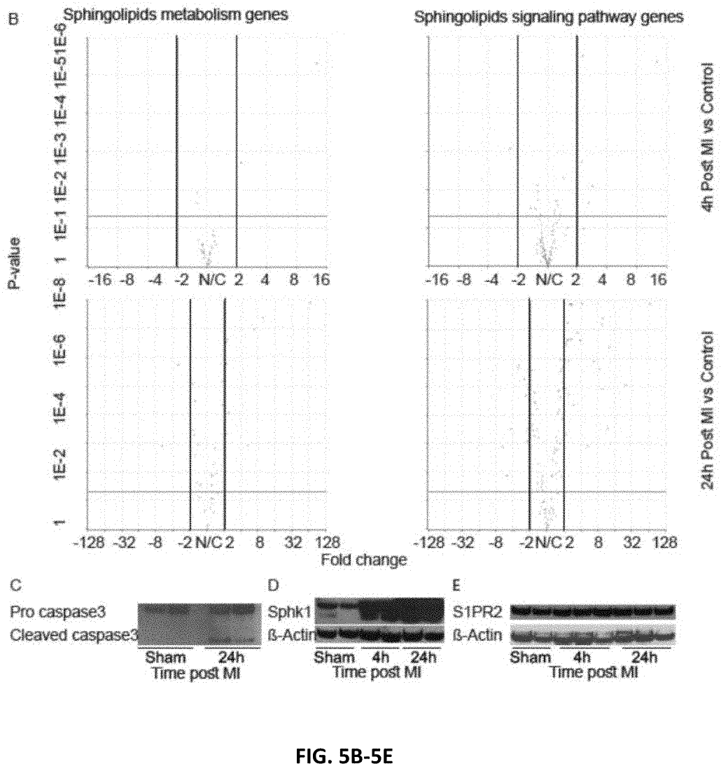

For cell death assessment the hearts were harvested at 1, 2, 4, and 28 days post MI and from sham operated mice. TUNEL stain was used to assess DNA fragmentation in cardiac cells Troponin-I immunostaining was used to distinguish between cardiomyocytes and non-cardiomyocytes (FIG. 1A). The highest level of DNA fragmentation was found 24 h post MI with 9±2% of total cells in LV has a fragmented DNA, 15±3% of CM and 4±0.2% of non CM. The levels of DNA fragmentation two days post MI reduced both in CM and non CM and reached to a basal levels 28 d post MI with 0.1±0.1% of total cells 0.07±0.08% of CM and 0.12±0.1% of non CM comparable to the levels in the hearts of control mice. Cleaved Caspase3 immunoblotting 24 h post MI confirmed high level of apoptosis in the infarcted area (FIG. 5C).

Sphingolipids metabolism and signaling pathway partial transcriptomes were studied in hearts of sham operated mice or mice 4 h and 24 h post MI. We focused on two partially overlapping sets of genes: Sphingolipid metabolism genes based on KEGG PATHWAY map00600 and Sphingolipid signaling pathway genes based on KEGG PATHWAY map04071[11]. In the Sphingolipids metabolism transcriptome 4 h post ligation 2 genes were significantly upregulated by more than 2 fold and one was downregulated by less than −2 fold. 24 h post MI 10 genes were significantly upregulated by more than 2 fold and 2 were downregulated by less than −2 fold. Total of 12 out of 49 genes (not shown). In the Sphingolipids signaling pathway transcriptome 4 h post ligation 5 genes were significantly upregulated by more than 2 fold and 2 were downregulated by less than −2 fold. 24 h post MI, 28 genes were significantly upregulated by more than 2 fold and 10 were downregulated by less than −2 fold totals of 38 out of 82 genes (FIG. 1B and FIG. 5)

The dendrograms of both transcriptomes (FIG. 1B and FIG. 5A) shows that the control group and the 4 h post MI group are clustered together while the 24 h post MI group is cluster as a separate group suggesting that the major alterations in sphingolipids metabolism and signaling pathway related genes expression occurs more than 4 h post MI.

In order to study the role of ceram ides metabolites on cell death and heart function post MI we chose to alter ceramide metabolism and signaling pathway by enhancing ceramide degradation and S1P synthesis. First we confirm the RNA-seq DATA for the main genes that are involved in this process namely: Acid ceramidase (AC), Sphingosine Kinase 1 (Sphk1) and Sphingosine-1-Phosphate Receptor 2 (S1PR2) by qPCR and western blot analysis of hearts from an independent experiment. In agreement with the results of the RNAseq analysis, the relative levels of AC mRNA didn't change significantly (FIG. 1B). The levels of AC precursor did not change however, the levels of AC α subunit and β subunit gradually increased during infarct development (FIG. 1C) The increase in α and β subunits is accompanied by an increase in the activity level of AC (FIG. 1D). The mRNA levels of Sphk1 increased by 6 and 35 times 4 h and 24 h respectively. Western blot analysis reviled a dramatic increase in the levels of Sphk1 protein 4 h and 24 h post MI (FIGS. 1B and 1C and FIG. 1D). The relative levels of S1PR2 mRNA decline by 50% 4 h post MI and return to normal after 24 h. The levels of S1PR2 did not change 4 h or 24 h post MI (FIG. 1B and FIG. 4E).

Next, we checked the effect of these genes on the viability of neonatal rat cardiomyocytes (nrCM) under anoxic conditions. To this aim, we used a synthetic modRNA that encode to the human AC, Sphk1 and S1PR2. The expression kinetics of proteins encoded by modRNA and its reduced immunogenicity (Sultana 2017) make modRNA an ideal vector to study the role of genes expression in acute conditions such as myocardial infarction. First, we checked the effect of modRNA transfection on the expression levels of the target proteins in Hek293 cells (sup. FIG. 2A) or nrCM (FIG. 2A). In both cases, the levels of the protein encoded by the transfect modRNA were elevated in the transfected cells compare to control cells. To induce apoptosis in nrCM the cells were transfer to anoxic condition 18 h after transfection. After 48 h in anoxia, there was an elevation of 44% in the number of apoptotic cells, however, overexpression of AC or Sphk1 reduced the level of apoptotic cells by 22% and 27% respectively compared to control (FIG. 2B). Overexpression of S1PR2 reduced the level of apoptosis by 10% however, this reduction was not statistically significant (FIG. 2B).

When the cells were transfected with a combination of genes an additive effect was observed. Overexpression of AC and Sphk1 reduce the number of apoptotic cells by 48% and overexpression of AC and S1PR2 together reduce apoptosis by 33%. Surprisingly, combining Sphk1 with S1PR2 or combining AC, Sphk1 and S1PR2 did not reduce the levels of apoptosis (FIG. 2C).

To study the effect of AC, Sphk1, and S1PR2 on cell death in LV after myocardial infarction, hearts were infarcted by ligation of the left anterior descending artery. Immediately after the LAD was ligated, 100 μg modRNA encoding to a control gene or gene of interest were injected to the myocardium of the left ventricle. After 48 h the hearts were harvests and the levels of DNA fragmentation was measured. Strikingly, overexpression of AC in the left ventricle immediately after LAD ligation reduced the number of cells with fragmented DNA in the left ventricle by 54% compare to hearts that were treated with Luc modRNA. Overexpression of Sphk1 reduced DNA fragmentation by 29% and S1PR2 did not prevent the fragmentation of DNA in the LV 48 h post-MI (FIG. 3B). When a combination of genes was injected to the LV immediately after LAD ligation, only the combination of AC and Sphk1 had a mild additive effect of 59% reduction. AC+S1PR2 reduce DNA fragmentation by 21% and AC+Sphk1+S1PR2 reduce DNA fragmentation by 22%. Unexpectedly, overexpression of Sphk1 and S1PR2 induced DNA fragmentation post MI by 30% compare to control (FIG. 3B).

The beneficial effects of AC and Sphk1 and the additive effect of the combined expression of these two genes prompted us to study their effect on heart remodeling and function post MI. To this aim, we injected AC, Sphk1, AC+Sphk1 or Luc directly to the LV and compare the Left ventricular internal dimension-diastole (LVIDd), Left ventricular internal dimension-systole (LVIDs) and fractioning shortening % (% FS) at different time point post MI. At the end of the experiment (29 days post MI) the hearts were harvested and immunostained with WGA and CD31 to assess the average area of cardiomyocytes and the number of vessels in the LV. To measure the scar size, Masson's trichrome stain was performed on heart sections. Two days post-MI, there was no significant difference between the groups in all measured parameters (FIG. 8A). However, 28 days post MI % FS of LV in mice that were treated with AC Sphk1 or AC+Sphk1 were 46.4% 45% and 46.1% respectively compared to 38.8% in control mice (FIG. 9A). The LVIDs of mice treated with AC Sphk1 or AC+Sphk1 were lower than in control mice—1.65 mm, 1.72 mm, and 1.57 mm respectively compare to 2.02 mm in control LVIDd of treated mice was not significantly different than the LVIDd of control mice except for mice treated with AC that showed mild reduction in LVIDd compare to control (FIG. 4C and D). Those results Indicates that injecting AC or Sphk1 to the LV during acute MI results in better heart function in treated mice compared to the control.

In accordance with the beneficial effect that AC and Sphk1 have on heart function we found a significant reduction in the scar size 29 days post MI. In mice treated with AC, Sphk1 or AC+Sphk1 the scar areas were 14.2%, 16.7% and 16.1% of LV area compared to 23.3% in control mice (FIG. 4D and FIG. 9C).

No signs of CM hypertrophy were found by WGA stain and no difference in the number of luminal structures in the LV could be observed by CD31 immunostaining (FIG. 8C and D).

To determine if the expression of AC and Sphk1 improves heart function by preventing apoptosis at early stages or by promoting heart regeneration after the infarct development we compare the % FS at 28 days post MI to the % FS 2 days post MI. Surprisingly we found that the heart function in mice that were treated with AC improved 28 days post MI compare to the heart function at 2 days post MI by 1.5% in average. In contrast, % FS in control mice reduced by 9%. In mice treated with Sphk1 and AC+Sphk1 there was very mild reduction in % FS of 0.8% and 0.3% respectively (FIG. 9B).

Given the fact that occasionally, some or all of the injected RNA is spilt to the LV rather than to the myocardium leading to reduction of the efficiency of cell transfection, we decided to examine the outcome of injection after excluding hearts that we suspect were not properly injected and can be classified as outliers. We identified 2 outliers in the AC group and 1 in the AC+Sphk1 group. After excluding those hearts from the statistics the improvement in % FS of AC hearts was in average 5.3% and in AC+Sphk1 hearts there was an average improvement of 2.7% (FIG. 4B). The % FS of AC and AC+Sphk1 after excluding outliers increased to 48.6 and 47.6 respectively (FIG. 4A) scar size in AC and AC+Sphk1 reduced to 12.4 and 14.5 respectively (FIG. 4E).

The survival rates of mice that were treated with AC modRNA were significantly higher than survival rates of control mice. 100% of the AC treated mice survived 90 days post MI while the survival rate of mice treated with control modRNA were 60%. The survival rates of mice treated with Sphk1 or AC+Sphk1 were 80% (FIG. 4F).

### Improving Cell Quality and Survival in Assisted Reproductive Technologies

Nowhere is the role of cell death more significant than in the field of reproduction. Ovulated oocytes undergo molecular changes characteristic of cell death unless successful fertilization occurs. Under normal physiological conditions 85-90% of oocytes succumb to cell death at some point during fetal or postnatal life. Clinically, when the remaining oocyte reserve has been exhausted (on average, this occurs in women around age 50), menopause ensues as a direct consequence of ovarian senescence. A major challenge of assisted reproduction technologies (ARTs) is to mimic the natural environment required to sustain oocyte and embryo survival.

Accordingly, the ability to increase cell quality and survival is of particular interest in reproductive cells, which have unique features, such as the ability of the oocyte to undergo a cortical reaction and triggering of protein expression in the fertilized zygote.

The formation of a human embryo starts with the fertilization of the oocyte by the sperm cell. This yields the zygote, which carries one copy each of the maternal and paternal genomes. To prevent fertilization by multiple sperm, the egg undergoes a cortical reaction; once a single sperm manages to penetrate the outer membrane of the oocyte, the oocyte develops a permanent, impermeable barrier.

Expression of the genetic information contained in the zygote starts only after the zygote divides a couple of times.

There are several studies that support association of the signaling lipid, ceramide, and its metabolizing enzymes with cellular and organismal aging. It has been reported that the intracellular level of ceramide increased during stress related signaling such as cell culture and aging. Ceramidase, for example, acid ceramidase (AC) is required to hydrolyze ceramide into sphingosine and free fatty acids. Sphingosine is rapidly converted to sphingosine-1-phosphate (S1P), another important signaling lipid that counteracts the effects of ceramide and promotes cell survival. Thus, AC is a “rheostat” that regulates the levels of ceramide and S1P in cells, and as such participates in the complex and delicate balance between death and survival.

We have previously shown that AC expression is carefully regulated during oocyte maturation and early embryo development (Eliyahu, et al, 2010). We have also found that the complete “knock-out” of AC function in mice leads to embryo death between the 2 and 8-cell stage (Eliyahu, FASEB J, 2007). In addition, our previous publication (Eliyahu, FASEB J, 2010) showed that the ceramide-metabolizing enzyme, AC is expressed and active in human cumulus cells and follicular fluid, essential components of this environment, and that the levels of this enzyme are positively correlated with the quality of human embryos formed in vitro. These observations led to a new approach for oocyte and embryo culture that markedly improves the outcome of in vitro fertilization (IVF).

The disclosed method provides an opportunity to improve egg quality. Firstly, when women have a failed IVF cycle or are considering undergoing IVF at an advanced maternal age, they are often told that they likely have poor-quality eggs. Why is egg quality so important for success in infertility treatment? The answer comes down to the simple fact that high-quality eggs produce high-quality embryos: 95% of embryo quality comes from the egg. Embryos must be strong enough to survive the early stages of development in order to result in a successful pregnancy.

As a woman ages, her ovaries' ability to produce high-quality eggs starts to decline. This is a condition known as diminished ovarian reserve (DOR) and is the most common cause of infertility for women over 40. Because of their poor egg quality (and resulting poor embryo quality), these women have difficulty conceiving on their own. Success rates of fertility treatments are also lower for these women, who are often refused treatment at fertility centers unless they are willing to use donor eggs. The method disclosed herein provides a treatment plan for these women geared toward improving the number and quality of eggs.

Ceramide has been shown to induce apoptotic cell death in different cells type [7] including murine and human cardiomyocytes [14, 15]. On the other hand, sphingosine, one of the products of ceramide degradation can be phosphorylated to give rise to a major agent of cell survival and cardioprotection sphingosine 1 phosphate [16, 17].

In this disclosure, we describe a strategy different from previously described approaches to reduce ceramide levels in the ischemic heart. Instead of targeting ceramide synthesis we study the effect of increasing ceramide degradation by overexpression of acid ceramidase. With this strategy, not only can we reduce ceramide levels but we also increase the reservoir of sphingosine which is the main building block for the pro survival molecule Sphingosine 1 phosphate. We found that modRNA encoding AC mediated high expression levels of AC in vitro and in vivo. This overexpression was accompanied with increased enzymatic activity in hearts post-MI and a reduce levels of apoptotic cells under an anoxic condition in vitro as well as reduce the number of cells with fragmented DNA in the left ventricle of mice 1 and 2 days post MI. We also observed reduce levels of Caspase3 in the hearts of AC treated hearts compared to mice treated with a control gene. 28 days post MI the function of hearts treated with AC was significantly better in terms of % FS LVIDd and LVIDs. When we compare % FS 28 d post-MI to % FS 2 d post-MI we observed a significant improvement which implies a better regenerative capacity in AC treated hearts. In consistent with reducing cell death and better heart function, the average scar size in AC treated mice was significantly smaller than the scar size of Luc control mice. Not only did we observe less apoptosis 1 and 2 days post MI and better heart functions 28 post MI, the survival rates of mice that were treated with AC modRNA were significantly higher than survival rates of control mice in a long-term survival assay suggesting a long-term effects of our intervention in the early events that are taking place as a results of acute MI.

Acid ceramidase catalyzes the hydrolysis of ceramide into sphingosine and free fatty acid [18]. While it has been reported that sphingosine is capable of disassembling mitochondrial ceramide channels suggesting the existence of an anti-apoptotic property of sphingosine [19, 20] other evidence support a positive role of sphingosine in the execution of apoptotic or necrotic cell death [21]. Moreover, it was suggested by Benaim et al [22] that Sphingosine can disturb the homeostasis of cellular calcium by inhibiting the activity of sarco(endo)plasmic reticulum Ca(2+)-ATPase (SERCA) which has a pivotal role in proper cardiac function [23, 24]. Two genes encode sphingosine kinase—Sphk1 and Sphk2. It catalyzes the phosphorylation of sphingosine to S1P and has been shown to possess cardioprotective properties [25]. Duan et al reported that adenoviral mediated overexpression of Sphk1 in rat hearts can protect the treated hearts from ischemia and reperfusion injury [26]. Our transcriptome analysis shows that the expression levels of Sphk1 are elevated by 12 and 67 fold 4 and 24 hours post-MI respectively. A similar trend was found with qPCR analyzed of Sphk1 levels in an independent experiment. This was accompanied by a significant elevation in Sphk1 protein levels as measured by western blot analysis. The pathway analysis of sphingosine signal transduction reviled an up regulation of all the components in the TNF signaling pathway including TNF alpha, TNFR, TRADD, and TRAF2. Interestingly, Xia et al showed that TRAF2 can interact with Sphk1 and that this interaction is necessary for the anti-apoptotic activity of TRAF2 [27]. Recently Guo et al reported a cardioprotective role of TRAF2 [28] It will be interesting to examine the role of Sphk1 and TRAF2 interaction in this context. In this study, modRNA mediated delivery of Sphk1 to isolated neonate rat cardiomyocytes, reduced the level of apoptosis in the transfected cells 48 h after the cell were transferred to an anoxic environment. Combined expression of Sphk1 and acid ceramidase in neonate rat cardiomyocytes had an additive effect demonstrating the importance of ceramide and sphingosine degradation as well as S1P synthesis in cardiomyocytes' survival. When Sphk1 was over express in infarcted hearts, we observed a nonsignificant reduction in the portion of nuclei with fragmented DNA in the left ventricle 48 h post-MI, 28 days post MI the % FS was moderately better in Sphk1 treated mice compare to Luc controls. The average scar size in Sphk1 mice was smaller than the average scar size in control mice; however the difference is not significant. The survival rates of Sphk1 treated mice were higher than the survival of control mice, however, the difference is not significant.

Contrary to the additive effect that was achieved by combined expression of AC and Sphk1 in isolated cardiomyocytes, in vivo we did not observe any advantage of combined expression over the effect we observed when the mice were treated AC in term of cell death 2 days' post-MI or heart function and scar size 28 days post MI. Not only has this but the survival rates of mice treated with AC and Sphk1 were not significantly lower than the survival rates of mice treated with AC alone. The above observation suggests that the high endogenous levels of Sphk1 during acute MI are sufficient in order to degrade sphingosine in the heart and keep it levels below toxic levels even in the presence of high AC levels. While the levels of Sphk1 after MI increased dramatically, the levels of Sphk2 are moderately reduced 4 h post-MI and then increased by less than 1.5 fold 24 h post-MI. Our unpublished data showed that overexpression of Sphk2 can reduce cell death in-vitro and in-vivo under stress condition however this effect was less significant than the positive effect of Sphk1 cell survival.

Sphingosine 1 phosphate exert its activity on cells by activating a family of five G protein-coupled receptors: S1pr1-5. The levels of the two most abundant receptors in the heart namely S1pr1 and 3 are moderately but significantly elevated after MI. In contrast, the levels of S1pr2 4 h after MI are reduced and 24 h post MI the levels are back to normal. The role of S1pr1 and S1pr3 in cardio protection is well established [25] however the role of S1p2 in heart function is less clear. Or results suggest that overexpression of S1p2 in cells and in heart have a neglected effect on cells survival.

### Cell Senescence

Senescence is the major cause of suffering, disease, and death in modern times. Senescence, or biological aging, is the slow drop of functional characteristics. Senescence can refer either to cellular senescence or to the senescence of a whole organism. In addition to induced senescence such as aging, there is stress-induced senescence, which is a very broad concept including a variety of stress conditions such as oxidative stress, injury, noise exposure, and other sources of damage to cells. These stresses act via intracellular pathways to induce a state of non-proliferation. Cellular senescence described by Hayflick and Moorhead in the 1960s, is the irreversible arrest of cells following long culture. Telomere shortening is the key mechanism driving replicative senescence in human fibroblasts. Apart from cell cycle arrest, senescent cells have been shown to experience dramatic changes in terms of gene expression, combination of CDK1 activity, heterochromatin formation, metabolism including (Sphingolipids metabolism), epigenetics, and a distinct secretion profile known as the Senescence-Associated Secretory Phenotype (SASP) (Copp'e et al., 2014). SASP is a way for senescent cells to communicate with the immune system, potentially to facilitate their own clearance (for example pro-inflammatory cytokines) and contribute to disruption of cell and tissue homeostasis and function (Shay and Wright, 2010). It has been shown that “chronic” SASP is able to induce senescence in adjacent young cells, contributing to tissue dysfunction (Acosta et al., 2013, Jurk et al., 2014). Senescent cells also show mitochondrial dysfunction (Passos et al., 2010).

Oxidative stress-induced senescence in the heart caused by myocardial infarction (MI) can trigger cardiomyocyte death or senescence (Huitong et al., 2018). Moreover, senescence can have deleterious effects with chronic, worsening pathologies such as type 2 diabetes (Palmer et al., 2015), atherosclerosis (Gorenne et al., 2006; Wang et al., 2015), Multiple Sclerosis (MS) (Oost et al., 2019), and other chronic diseases.

The involvement of sphingolipids has been studied in multiple organisms and cell types for the regulation of aging and senescence, especially ceramide and sphingosine-1-phosphate (S1P) for induced cellular senescence, distinct from their effect on survival. Significant and wide-ranging evidence defines critical roles of sphingolipid enzymes and pathways in aging and organ injury leading to tissue senescence (Trayssac et al., 2018), including regulation by stress stimuli, p53, participation in growth arrest, SASP, and other aspects of the senescence response. Acid ceramidase is the only protein that can balance the level of ceramide vs S1P by hydrolyzing ceramide to a product that can be phosphorylated to form S1P. The present invention is based on the further discovery that in addition to its role in protecting cells from apoptosis, administration of AC decreased the rate of senescence in vitro, and in vivo, in different cell types and tissues.

Blockage in the coronary arteries reduces the supply of blood to heart muscle and causes dynamic effects within the infarction risk area and around the ischemic border zone. Tissues in the infarction risk area exhibit distinct metabolic changes within a few minutes. Nearly the entire risk area tissues become irreversibly injured during a severe hypoperfusion of 6 hours. On the other hand, the border zone tissues exhibit only moderate metabolic changes due to greater collateral perfusion, including from 45-80% of blood flow regionally in the non-ischemic vascular bed. The ischemic border zone tissues are from the lateral edges of infarct, are approximately 2 mm wide, and increase in width along the subepicardium. Over time, the subepicardial margins of border zone widen due to improved collateral blood flow. The tissues in the border zone region are in, or entering into, senescence.

### EXAMPLES

### Mice

All animal procedures were performed under protocols approved by the Icahn School of Medicine at Mount Sinai Institutional Care and Use Committee. CFW mice strains, male and female, were used for studies on heart function following myocardial infarction. Before surgery mice were anaesthetized with ketamine 100 mg/kg and xylazine 10 mg/kg cocktail.

For protein expression assay, 100 μg of Luc, Sphk1 or S1pr2 modRNA in 60 μl citrate sucrose buffer (sultana 2017) were injected directly into the myocardium in an open-chest surgery. MI was induced by permanent ligation of the LAD. The left thoracic region was shaved and sterilized. After intubation, the heart was exposed through a left thoracotomy. A suture was placed to ligate the LAD. When needed, 100 μg modRNA was injected into the infarct border zone immediately after LAD ligation. The thoracotomy and skin were sutured closed in layers. Excess air was removed from the thoracic cavity, and the mouse was removed from ventilation when normal breathing was established. Hearts from sham operated mice were collected immediately after open chest operation without LAD ligation.

### Synthesis of modRNA

Clean PCR products generated with plasmid templates served as template for mRNA. ModRNAs were transcribed in vitro using a custom ribonucleoside blend of Anti Reverse Cap Analog, 3′-O—Me-m7G(5′) ppp(5′)G (6 mM, TriLink Biotechnologies), guanosine triphosphate (1.5 mM, Life Technologies), adenosine triphosphate (7.5 mM, Life Technologies), cytidine triphosphate (7.5 mM, Life Technologies), N1-Methylpseu-douridine-5′-Triphosphate (7.5 mM, TriLink Biotechnologies). The mRNA was purified using a Megaclear kit (Life Technologies) and was treated with Antarctic Phosphatase (New England Biolabs); then it was purified again using the Megaclear kit. The mRNA was quantitated by Nanodrop (Thermo Scientific), precipitated with ethanol and ammonium acetate, and resuspended in 10 mM TrisHCl and 1 mM EDTA.

### In Vitro Transfection of modRNA in Cardiomyocytes

2.5 ug per well (of 24 well plate) of mRNA encoding nGFP, AC, Sphk1 or S1pr2 were complexed with RNAiMAX (Life Technologies) and transfected into neonatal rat or hPSC-derived CMs according to the manufacturer's instructions. For Immunofluorescent staining, 18 hr post-transfection cells were washed 1 time with PBS fixed with 4% PFA for 10 min and washed 3 times with PBS. For western blot analysis cells were washed 1 time with PBS and then lysed with lysis buffer (Sigma). For anoxic induced apoptosis, cells were transfer to anoxia chamber for 48 h and harvested with Trypsin 0.25% (Sigma) for FACS analysis.

### In Sperm and Oocytes

Mouse sperm and oocytes were treated with 50 to 200 ng/microliter of naked AC modRNA into the culture media. In some embodiments, 100 ng/plwas used. Pronuclei (PN) embryos can be injected with modRNA by intracytoplasmic injection. In some embodiments, embryos were injected with 50-100 ng of modRNA.

### hPSC Differentiation

For heart function following myocardial infarction studies, hPSCs (H9) were differentiated along a cardiac lineage as previously described. Briefly, hPSCs were maintained in E8 media and passaged every 4-5 days onto matrigel-coated plates. To generate embryonic bodies (EBs), hPSCs were treated with 1 mg/ml collagenase B (Roche) for 30 min or until cells dissociated from plates. Cells were collected and centrifuged at 1,300 rpm for 3 min, and they were resuspended into small clusters of 50-100 cells by gentle pipetting in differentiation media containing RPMI (Gibco), 2 mmol/L L-glutamine (Invitrogen), 4 x 10 monothioglycerol (MTG, Sigma), 50 mg/mL ascorbic acid (Sigma), and 150 mg/mL transferrin (Roche). Differentiation media were supplemented with 2 ng/mL BMP4 and 3 mmol Thiazovivin (Millipore) (day 0). EBs were maintained in six-well ultra-low attachment plates (Corning) at 37° C. in 5% CO2, 5% O2, and 90% N2. On day 1, media were changed to differentiation media supplemented with 20 ng/mL BMP4 (R&D Systems) and 20 ng/mL Activin A (R&D Systems). On day 4, media were changed to differentiation media supplemented with 5 ng/mL VEGF (R&D Systems) and 5 mmol/L XAV (Stemgent). After day 8, media were changed every 5 days to differentiation media without supplements.

### Neonatal Rat CM Isolation

Neonatal rat ventricular CMs were isolated from 3- to 4-day-old Sprague-Dawley rats (Jackson ImmunoResearch Labora-tories). We used multiple rounds of digestion with 0.1% collagenase II (Invitrogen) in BPS. After each digestion, the supernatant was collected in horse serum (Invitrogen). Total cell suspension was centrifuged at 1,500 rpm for 5 min. Supernatants were discarded and cells were resuspended in DMEM (Gibco) with 0.1 mM ascorbic acid (Sigma), 0.5% Insulin-Transferrin-Selenium (100×), penicillin (100 U/mL), and streptomycin (100 mg/mL). Cells were plated in plastic culture dishes for 90 min until most of the non-myocytes attached to the dish and myocytes remained in the suspension. Myocytes were then seeded at 1×10 cells/well in a 24-well plate. Neonatal rat CMs were incubated for 48 hr in DMEM containing 5% horse serum. After incubation, cells were transfected with modRNAs as described above.

### Real-Time qPCR Analyses

Total RNA was isolated using the RNeasy mini kit (QIAGEN) and reverse transcribed using Superscript III reverse transcriptase (Invitrogen), according to the manufacturer's instructions. Real-time qPCR analyses were performed on a Mastercycler realplex 4 Sequence Detector (Eppendoff) using SYBR Green (Quantitect™ SYBR Green PCR Kit, QIAGEN). Data were normalized to 18srRNA expression where appropriate (endogenous controls). Fold changes of gene expression were determined by the ddCT method. PCR primer sequences are summarized in Table 2.

TABLE 2

| | | SEQ ID | | SEQ ID |
|---|---|---|---|---|
| Gene | Forward | NO. | Reverse | NO. |
| AC | ACAGGATTCAAACCAGGACTGT | 19 | TGGGCATCTTTCCTTCCGAA | 20 |
| AC | TGACAGGATTCAAACCAGGACT | 21 | CTGGGCATCTTTCCTTCCGA | 22 |
| Sphk1 | ATACTCACCGAACGGAAGAACC | 23 | CCATTAGCCCATTCACCACC | 24 |
| | | | TC | |
| Sphk1 | ACTGATACTCACCGAACGGAA | 25 | CATTAGCCCATTCACCACCT | 26 |
| | | | C | |
| S1PR2 | CACAGCCAACAGTCTCCAAA | 27 | TCTGAGTATAAGCCGCCCA | 28 |
| S1PR2 | ATAGACCGAGCACAGCCAA | 29 | GAACCTTCTCAGGATTGAGG | 30 |
| | | | T | |
| 18s rRNA* | TAACGAACGAGACTCTGGCAT | 31 | CGGACATCTAAGGGCATCAC | 32 |
| | | | AG | |

*Genetic Vaccines and Therapy 2004, 2:5

### Western Blot

Upon thawing, hearts lysates' were subjected to separation by SDS-PAGE using 12% precast Nupage Bis/Tris gels (Invitrogen, Carlsbad, Calif., USA) under reducing conditions and MES running buffer (Invitrogen), and transferred onto a nitrocellulose membrane (Bio-Rad) using a semidry transfer apparatus and Nupage-MOPS transfer buffer (Invitrogen). The membrane was block with TBS/Tween containing 5% dry milk and incubated with specific primary antibodies over night at 4 OC washed with TBS/Tween and incubated with rabbit or gout antibodies conjugated to hors reddish peroxidase for 1 hour at room temperature. Detection was performed by an enhanced chemiluminecence (ECL) detection system (Pierce, Rockford, Ill.). For molecular weight was determination, using prestained protein standards (Amersham, Buckinghamshire, UK).

### Immunohistochemistry

The mouse hearts were harvested and perfused using perfusion buffer (2 g/l butanedione, monoxime and 7.4 g/l KCl in PBS×1) and 4% paraformaldehyde (PFA). Hearts were fixed in 4% PFA/PBS overnight on shaker and then washed with PBS for 1 hr and incubated in 30% sucrose/PBS at 4 O C overnight. Before freezing, hearts were mounted in OCT for 30 min and frozen at −80° C. Transverse heart sections of 10 μM were made by cryostat. Cryosections were washed in PBST and blocked for 1 h with 5% donkey serum in PBST. Sections were incubated over night at 4° C. using primary antibodies for Troponin I, Sphk1, S1p2. Secondary antibodies were used for fluorescent labeling (Jackson ImmunoResearch Laboratories). TUNEL staining was performed according to manufacturer's recommendations (In-Situ Cell Death Detection Kit, Fluorescein, Cat #11684795910, Roche). Stained sections were imaged using a Zeiss Slide Scanner Axio Scan or Zeiss mic. Quantification of TUNEL in cardiac sections was performed using ImageJ software. For cell immunocytochemistry, Hek293 and isolated CMs were fixed on coverslips with 4% PFA for 10 min at room temperature. Following permeabilization with 0.1% TRITON® X100 in PBS for 10 min at room temperature, cells were blocked with 5% Donkey serum+0.1% TRITON® X100 in PBS for 30 minutes. Coverslips were incubated with primary antibodies in humidity chamber for 1 hour at room temperature followed by incubation with corresponding secondary antibodies conjugated to Alexa Fluor 488, Alexa Fluor 647 and Alexa Fluor 555, and Hoechst 33342 staining for nuclei visualization (all from Invitrogene). The fluorescent images were taken on a Zeiss fluorescent microscope at 20× magnification.

### Methods for Assisted Reproduction Studies

### Mouse Oocyte and Sperm Collection

All experiments involving animals were approved by and performed in strict accordance with the guidelines of the appropriate institutional animal care and use committees. Seven- to 8-wk-old 129-SVIMJ and C57-Black/6 female mice (Jackson Laboratory, Bar Harbor, Me.) were superovulated with 10 IU of pregnant mare serum gonadotropin (PMSG; Syncro-part, Sanofi, France), followed by 10 IU of human chorionic gonadotropin (hCG; Sigma, St. Louis, Mo.) 48 hours later. Mature and aged MII oocytes were collected from the oviduct ampullae at 16 or 46 hour after injection of hCG, respectively. Cumulus cells were removed by a brief exposure to 400 IU/ml of highly purified hyaluronidase (H-3631; Sigma) in M2 medium (Sigma). Epididymal sperm from 10-wk-old mice were used for IVF of oocytes from the same strain.

### Mouse Fertilization and Embryo Culture

Microdrops of fertile sperm in Vitrofert solution (Vitrolife, Goteborg, Sweden) were prepared, and ˜10 oocytes were placed into each sperm microdrop. The fertilization process was performed for 6 hours at 37° C. in a humidified atmosphere of 5% CO₂ and 95% air. After IVF, zygotes were washed 3 times with potassium simplex optimized medium (KSOM, Chemicon, Billerica MA) and cultured for an additional 20-48 hours at 37° C. in a humidified atmosphere of 5% CO₂ and 95% air. Cleavage of the zygotes was observed and recorded throughout the in vitro culture.

### Harvest, Evaluation and Culture of Human Gametes

### (A) Oocytes

Female patients undergo approved and controlled ovarian stimulation by administration of recombinant follicle-stimulating hormone (rFSH) followed by concomitant administration of gonadotropin-releasing hormone (GnRH) antagonist. Specifically, rFSH was administrated beginning from a day equal to ½ of the cycle. GnRH antagonist was added at day 6, or when follicles were 12 mm in diameter and until the leading follicle exceeds mm or the estradiol level is above 450 pg/ml. This protocol was continued until at least 2 follicles of 17-18 mm were observed. At this point, ovulation was induced by double trigger administration of Ovitrelle (LH) and Decapeptide (GnRH analogue). Ovum pickup was performed 36-38 h afterwards.

The cumulus-oocyte complexes was isolated into fertilization medium (LifeGlobal), in the presence of 100 μg/μl of AC modRNA.

### (B) Sperm

Sperm samples were evaluated for their count, motility and morphology, and all parameters were documented. Post validation sperm were incubated with Multipurpose Handling Medium® (MHM®, Irvine Scientific), and divided into two halves; one half was incubated in the presence of 100 ug/ul of AC modRNA in the media for 1 hour as the study group, and the second half was incubated in the absence of AC modRNA in the media for control. After a 1 hour incubation, a second evaluation of sperm samples for their count, motility and morphology was conducted. Values were compared to those obtained before treatment with AC modRNA.

Following incubation and evaluation, gametes were handled by an approved and common protocol. Oocytes were inseminated, or injected, by ICSI (intracytoplamic sperm injection) according to the spouse sperm parameters and routine protocol. After insemination, ICSI oocytes were transferred to Global medium (medium for culture of Life Global) as is routine in IVF/ICSI. All embryos were incubated and embryonic development was monitored from the time of fertilization up to day 5 in the integrated EmbryoScope™ time-lapse monitoring system (EMBRYOSCOPE™, UnisenseFertiliTech, Vitrolyfe Denmark). The EMBRYOSCOPE™ offers the possibility of continuous monitoring of embryo development without disturbing culture conditions. Embryo scoring and selection with time-lapse monitoring was performed by analysis of time-lapse images of each embryo with software developed specifically for image analysis (EmbryoViewer workstation; UnisenseFertilitech A/S). Embryo morphology and developmental events were recorded to demonstrate the precise timing of the observed cell divisions in correlation to the timing of fertilization as follows: time of 1) pronuclei fading (tPnf), 2) cleavage to a 2-blastomere (t2), 3) 3-blastomere (t3), 4) 4-blastomere (t4) and so forth until reaching an 8-blastomere (t8) embryo, 5) compaction (tm), and 6) start of blastulation. In addition, the synchrony and the duration of cleavages were also measured. Blastocyst morphology including the composition of the inner cell mass and the trophectoderm, were evaluated according to the Gardner blastocyst grading scale.

Preimplantation genetic screening (PGS) is performed by chromosomal microarray analysis (CMA) in order to select euploid embryos for transfer. For this, trophectoderm biopsy is performed on day 5. Subsequently, blastocysts and the biopsied embryos are frozen by vitrification. DNA from trophectodermal samples is subjected to whole genome amplification (WGA) and CMA as previously described (Frumkin et al., 2017). Embryos found to be euploid are thawed in a subsequent cycle and transferred to the uterus of the mother for implantation and pregnancy.

Following fertilization, the number of mouse and bovine embryos formed in the presence of AC also was improved (from approximately 40 to 88%), leading to approximately 5-fold more healthy births. Significantly more high-grade blastocysts were formed, and the number of morphologically intact, hatched embryos was increased from approximately 24 to 70% (Eliyahu et al., 2010).

During an IVF protocol, embryo culture can last up to 7 days and the chance of embryo survival is low especially for early embryos produced by aged oocytes. As shown in Table 3 mouse oocytes aged in vitro (that serve as a model for oocyte of elderly woman's) have higher chances to develop in to healthy embryos post AC treatment (Fertilization rate increased from 0.02% to 25.2%) (Eliyahu et al., 2010). Since the embryo's gene activation machinery is not fully functional yet, it's very challenging for the embryos to survive for so long in culture.

As part of our effort to prolong embryo survival in culture we developed a method for preventing the apoptotic death of embryos cultured in vitro by administering an effective amount of the sphingolipid-metabolizing protein, acid ceramidase-encoding modRNA. The present disclosure describes using modRNA rather than recombinant protein based on the observation that modRNA can supply enzyme expression for at least 10 days even post embryo transfer and implantation. Usually, during human IVF protocol embryos will be transferred between days 3-5 and it is not possible to expose the embryo post transfer to the recombinant protein. In addition, all embryos will be incubated from the time of fertilization up to day 5 in the integrated EmbryoScopeTM time-lapse monitoring system (EmbryoScope™, UnisenseFertiliTech, Vitrolyfe Denmark). The EmbryoScope™ offers the possibility of continuous monitoring of embryo development without disturbing culture conditions. The use of recombinant protein requires disruption of culture condition in order to refresh the media every 24-48 h.

Preliminary results demonstrated that modRNA “survival cocktail” injection into early mouse embryos dramatically improves the number of formed blastocytes (Table 3) and the number of live-born pups during IVF and embryo injection (Table 4).

TABLE 3

| | Zygotes | | |
|---|---|---|---|
| Conditions | Number | 2 cells | Blastocytes |
| Control | 81 | 70/81 | 27/81 |
| | | (86%) | (33%) |
| AC ModRNA | 101 | 91/101 | 78/101 |
| | | (90%) | (77%) |

AC ModRNA improves the quality of embryos cultured in vitro. Mice sperm were incubated with 100ng/u Naked AC ModRNA for 1 h in 37° C. CO₂ incubator. Post incubation, sperm were used for standard insemination (IVF) of C57BL/6 MII eggs. *(P<0.003).

TABLE 4

| | | | 2-4 | | |
|---|---|---|---|---|---|
| Conditions | oocytes | Zygotes | cells | Pups | Pups/Oocytes |
| Control | 107 | 86 | 72 | 8 | 8/107 = 7.5% |
| AC ModRNA | 116 | 98 | 91 | 19 | 19/116 = 16.4% |

AC ModRNA improves birth rate. Mice sperm were incubated with 100 ng/ul Naked AC ModRNA for 1 h in 37° C. CO₂ incubator. Post incubation, sperms were used for standard insemination (IVF) of C57BL/6 MII eggs. All of the embryos from both groups were then transferred into pseudo pregnant female recipients, and the birth rates were recorded. As shown in Table 4, the birth rate of implanted 2- to 4-cell embryos from the AC ModRNA treated group (8/86, 19%) was higher than that without treatment (8/86, 9%), indicating no deleterious effect of the AC ModRNA treatment on implantation or development. The pups derived from the rAC-treated embryos were followed for up to 1 month, and all had a normal appearance and motor function (data not shown). *(P<0.05).

Survival effect of AC modRNA is evaluated on the basis of 1) sperm parameters, 2) embryo morphology and morphokinetics from day 1-5, 3) blastocyst ploidy, and 4) pregnancy rate.

Overall, these data identify AC as an important component of the in vivo/in vitro oocyte and embryo environment, and provide a novel technology for enhancing the outcome of assisted fertilization.

### REFERENCES

Perez G I, Tao X J, Tilly J L. Fragmentation and death (a.k.a. apoptosis) of ovulated oocytes. Mol Hum Reprod. 1999; 5 (5):414-20.
Eliyahu E, Park J H, Shtraizent N, He X, Schuchman EH. Acid ceramidase is a novel factor required for early embryo survival. FASEB J. 2007; 21 (7):1403-9.
Eliyahu E, Shtraizent N, Martinuzzi K, Barritt J, He X, Wei H, Chaubal S, Copperman A B, Schuchman E H. Acid ceramidase improves the quality of oocytes and embryos and the outcome of in vitro fertilization. FASEB J. 2010; 24 (4):1229-38.
Katalin Karikó, Hiromi Muramatsu, Frank A Welsh, János Ludwig, Hiroki Kato, Shizuo Akira, Drew Weissman. Incorporation of Pseudouridine Into mRNA Yields Superior Nonimmunogenic Vector With Increased Translational Capacity and Biological Stability. Mol Ther. 2008; 16 (11): 1833-1840.
Yang H, Wang H, Shivalila C S, Cheng A W, Shi L, Jaenisch R. One-step generation of mice carrying reporter and conditional alleles by CRISPR/Cas-mediated genome engineering. Cell. 2013; 154 (6):1370-9.
Wu Y, Liang D, Wang Y, Bai M, Tang W, Bao S, Yan Z, Li D, Li J. Correction of a genetic disease in mouse via use of CRISPR-Cas9. Cell Stem Cell. 2013; 13 (6):659-62.
Ruzo A, Brivanlou A H. At Last: Gene Editing in Human Embryos to Understand Human Development. Cell Stem Cell. 2017; 21 (5):564-565.
Frumkin T, Peleg S, Gold V, Reches A, Asaf S, Azem F, Ben-Yosef D, Malcov M. Complex chromosomal rearrangement-a lesson learned from PGS. J Assist Reprod Genet. 2017; 34 (8):1095-1100.

1. A method to inhibit cell death and/or cell senescence and/or promote survival of a mammalian cell or group of mammalian cells, comprising contacting said cell or cells with a modified RNA (modRNA) that encodes a sphingolipid-metabolizing protein.

2. The method of claim 1, wherein said sphingolipid-metabolizing protein is selected from the group consisting of (1) a ceramidase; (2) a sphingosine kinase (SPHK); (3) sphingosine-1-phosphate receptor (SIPR) or a combination of (1), (2), and (3), a combination of (1) and (2), a combination of (1) and (3), or a combination of (2) and (3).

3. The method of claim 1, wherein said mammalian cell or group of mammalian cells are selected from the group consisting of primary cells, stems cells and gametes.

4. The method of claim 3, wherein said mammalian cell or group of mammalian cells is selected from the group consisting of cardiac cells, muscle cells, epithelial cells, endothelial cells, oocytes, sperm, and embryos.

5. A composition comprising (1) a modRNA that encodes a ceramidase; (2) a modRNA that encodes sphingosine kinase (SPHK); (3) a modRNA that encodes sphingosine-1-phosphate receptor (SIPR) or a combination of (1), (2), and (3), a combination of (1) and (2), a combination of (1) and (3), or a combination of (2) and (3) and a pharmaceutically acceptable carrier.

6. The method of claim 1, wherein the sphingolipid-metabolizing protein is a ceramidase.

7. The method of claim 6, wherein the ceramidase is an acid ceramidase.

8. The method of claim 7, wherein the acid ceramidase has the nucleotide sequence of SEQ ID NO: 1 or SEQ ID NO: 7.

9. The method of claim 1, wherein the ceramidase is an alkaline ceramidase.

10. The method of claim 9, wherein the alkaline ceramidase has the nucleotide sequence of SEQ ID NO: 13, SEQ ID NO: 14, SEQ ID NO: 15, SEQ ID NO: 16 or SEQ ID NO: 17.

11. The method of claim 1, wherein the ceramidase is a neutral ceramidase.

12. The method of claim 11, of claim 5, wherein the neutral ceramidase has the nucleotide sequence of SEQ ID NO: 6, SEQ ID NO: 8, SEQ ID NO: 9, SEQ ID NO: 10, SEQ ID NO: 11 or SEQ ID NO: 12.

13. The method of claim 1, wherein the sphingolipid-metabolizing protein is sphingosine kinase (SPHK).

14. The method of claim 1, wherein the SPHK has the nucleotide sequence of SEQ ID NO: 2.

15. The method of claim 1, wherein said sphingolipid-metabolizing protein is S1PR2.

16. The method of claim 15, wherein the S1PR2 has the nucleotide sequence of SEQ ID NO: 3.

17. The method of claim 1, wherein said cells or group of cells are contacted with modRNA that encodes a ceramidase and modRNA that encodes sphingosine kinase (SPHK).

18. The method of claim 1, wherein said cells or group of cells are contacted with modRNA that encodes a ceramidase, modRNA that encodes sphingosine kinase (SPHK) and modRNA that encodes sphingosine-1-phosphate receptor (SIPR).

19. The method of claim 1, wherein said cells or group of cells are contacted with modRNA that encodes a ceramidase and modRNA that encodes sphingosine-1-phosphate receptor (SIPR).

20. The method of claim 17, wherein said ceramidase is acid ceramidase (AC).


SEQUENCE LISTING (32 sequences)

SEQ ID NO: 1 - DNA, 1188 bp, Artificial Sequence
  synthetic oligonucleotide
atgccgggccggagttgcgtcgccttagtcctcctggctgccgccgtcagctgtgccgtcgcgcagcacgcgccgccgtggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 2 - DNA, 1194 bp, artificial sequence
  synthetic nucleotide
atccagtggtcggttgcggacgtggcctctttggttttgttttctcagcgggcggcccccggggcgtgctcccgcggccctgccgcgtgctggtgctgctgaacccgcgcggcggcaagggcaaggccttgcagctcttccggagtcacgtgcagccccttttggctgaggctgaaatctccttcacgctgatgctcactgagcggcggaaccacgcgcgggagctggtgcggtcggaggagctgggccgctgggacgctctggtggtcatgtctggagacgggctgatgcacgaggtggtgaacgggctcatggagcggcctgactgggagaccgccatccagaagcccctgtgtagcctcccagcaggctctggcaacgcgctggcagcttccttgaaccattatgctggctatgagcaggtcaccaatgaagacctcctgaccaactgcacgctattgctgtgccgccggctgctgtcacccatgaacctgctgtctctgcacacggcttcggggctgcgcctcttctctgtgctcagcctggcctggggcttcattgctgatgtggacctagagagtgagaagtatcggcgtctgggggagatgcgcttcactctgggcaccttcctgcgtctggcagccctgcgcacctaccgcggccgactggcctacctccctgtaggaagagtgggttccaagacacctgcctcccccgttgtggtccagcagggcccggtagatgcacaccttgtgccactggaggagccagtgccctctcactggacagtggtgcccgacgaggactttgtgctagtcctggcactgctgcactcgcacctgggcagtgagatgtttgctgcacccatgggccgctgtgcagctggcgtcatgcatctgttctacgtgcgggcgggagtgtctcgtgccatgctgctgcgcctcttcctggccatggagaagggcaggcatatggagtatgaatgcccctacttggtatatgtgcccgtggtcgccttccgcttggagcccaaggatgggaaaggtgtgtttgcagtggatggggaattgatggttagcgaggccgtgcagggccaggtgcacccaaactacttctggatggtcagcggttgcgtggagcccccgcccagctggaagccccagcagatgccaccgccagaagagcccttatatg

SEQ ID NO: 3 - DNA, 1062 bp, Artificial sequence
  synthetic nucleotide
atgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgccttcatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggaggccgggggtgggggtgcaaggacggaggcggggcgggaccccgggccaccacctcctgccactccgcagctccagctccctggagaggggcatgcacatgcccacgtcacccacgtttctggagggcaacacggtggtcatg

SEQ ID NO: 4 - DNA, 1653 bp, Artificial Sequence
  synthetic nucleotide
atggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaa

SEQ ID NO: 5 - DNA, 801 bp, Artificial Sequence
  synthetic nucleotide
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggagatccaaaaaagaagagaaaggtaggcgatccaaaaaagaagagaaaggtaggtgatccaaaaaagaagagaaaggtataa

SEQ ID NO: 6 - DNA, 1236 bp, Artificial Sequence
  synthetic nucleotide
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaacgtacagaggtgcagttccatggtacaccataaatcttgacttaccaccctacaaaagatggcatgaattgatgcttgacaaggcaccagtgctaaaggttatagtgaattctctgaagaatatgataaatacattcgtgccaagtggaaaaattatgcaggtggtggatgaaaaattgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 7 - DNA, 1170 bp, Artificial Sequence
  synthetic nucleotide
atgaactgctgcatcgggctgggagagaaagctcgcgggtcccaccgggcctcctacccaagtctcagcgcgcttttcaccgaggcctcaattctgggatttggcagctttgctgtgaaagcccaatggacagaggactgcagaaaatcaacctatcctccttcaggaccaactgtcttccctgctgttataaggtacagaggtgcagttccatggtacaccataaatcttgacttaccaccctacaaaagatggcatgaattgatgcttgacaaggcaccagtgcctggcctacttggcaactttcctggcccttttgaagaggaaatgaagggtattgccgctgttactgatatacctttaggagagattatttcattcaatattttttatgaattatttaccatttgtacttcaatagtagcagaagacaaaaaaggtcatctaatacatgggagaaacatggattttggagtatttcttgggtggaacataaataatgatacctgggtcataactgagcaactaaaacctttaacagtgaatttggatttccaaagaaacaacaaaactgtcttcaaggcttcaagctttgctggctatgtgggcatgttaacaggattcaaaccaggactgttcagtcttacactgaatgaacgtttcagtataaatggtggttatctgggtattctagaatggattctgggaaagaaagatgtcatgtggatagggttcctcactagaacagttctggaaaatagcacaagttatgaagaagccaagaatttattgaccaagaccaagatattggccccagcctactttatcctgggaggcaaccagtctggggaaggttgtgtgattacacgagacagaaaggaatcattggatgtatatgaactcgatgctaagcagggtagatggtatgtggtacaaacaaattatgaccgttggaaacatcccttcttccttgatgatcgcagaacgcctgcaaagatgtgtctgaaccgcaccagccaagagaatatctcatttgaaaccatgtatgatgtcctgtcaacaaaacctgtcctcaacaagctgaccgtatacacaaccttgatagatgttaccaaaggtcaattcgaaacttacctgcgggactgccctgacccttgtataggttggtga

SEQ ID NO: 8 - DNA, 2346 bp, Artificial Sequence
  synthetic nucleotide
atggccaaacgcaccttctctaacttggagacattcctgattttcctccttgtaatgatgagtgccatcacagtggcccttctcagcctcttgtttatcaccagtgggaccattgaaaaccacaaagatttaggaggccattttttttcaaccacccaaagccctccagccacccagggctccacagctgcccaacgctccacagccacccagcattccacagccacccagagctccacagccactcaaacttctccagtgcctttaaccccagagtctcctctatttcagaacttcagtggctaccatattggtgttggacgagctgactgcacaggacaagtagcagatatcaatttgatgggctatggcaaatccggccagaatgcacagggcatcctcaccaggctatacagtcgtgccttcatcatggcagaacctgatgggtccaatcgaacagtgtttgtcagcatcgacataggcatggtatcacaaaggctcaggctggaggtcctgaacagactgcagagtaaatatggctccctgtacagaagagataatgtcatcctgagtggcactcacactcattcaggtcctgcaggatatttccagtataccgtgtttgtaattgccagtgaaggatttagcaatcaaacttttcagcacatggtcactggtatcttgaagagcattgacatagcacacacaaatatgaaaccaggcaaaatcttcatcaataaaggaaatgtggatggtgtgcagatcaacagaagtccgtattcttaccttcaaaatccgcagtcagagagagcaaggtattcttcaaatacagacaaggaaatgatagttttgaaaatggtagatttgaatggagatgacttgggccttatcagctggtttgccatccacccggtcagcatgaacaacagtaaccatcttgtaaacagtgacaatgtgggctatgcatcttacctgcttgagcaagagaagaacaaaggatatctacctggacaggggccatttgtagcagcctttgcttcatcaaacctaggagatgtgtcccccaacattcttggaccacgttgcatcaacacaggagagtcctgtgataacgccaatagcacttgtcccattggtgggcctagcatgtgcattgctaagggacctggacaggatatgtttgacagcacacaaattataggacgggccatgtatcagagagcaaaggaactctatgcctctgcctcccaggaggtaacaggaccactggcttcagcacaccagtgggtggatatgacagatgtgactgtctggctcaattccacacatgcatcaaaaacatgtaaaccagcattgggctacagttttgcagctggcactattgatggagttggaggcctcaattttacacaggggaaaacagaaggggatccattttgggacaccattcgggaccagatcctgggaaagccatctgaagaaattaaagaatgtcataaaccaaagcccatccttcttcacaccggagaactatcaaaacctcacccctggcatccagacattgttgatgttcagattattacccttgggtccttggccataactgccatccccggggagtttacgaccatgtctggacgaagacttcgagaggcagttcaagcagaatttgcatctcatgggatgcagaacatgactgttgttatttcaggtctatgcaacgtctatacacattacattaccacttatgaagaataccaggctcagcgatatgaggcagcatcgacaatttatggaccgcacacattatctgcttacattcagctcttcagaaaccttgctaaggctattgctacggacacggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 9 - DNA, 2241 bp, Artificial Sequence
  synthetic nucleotide
atggccaaacgcaccttctctaacttggagacattcctgattttcctccttgtaatgatgagtgccatcacagtggcccttctcagcctcttgtttatcaccagtgggaccattgaaaaccacaaagatttaggaggccattttttttcaaccacccaaagccctccagccacccagggctccacagctgcccaacgctccacagccacccagcattccacagccacccagagctccacagccactcaaacttctccagtgcctttaaccccagagtctcctctatttcagaacttcagtggctaccatattggtgttggacgagctgactgcacaggacaagtagcagatatcaatttgatgggctatggcaaatccggccagaatgcacagggcatcctcaccaggctatacagtcgtgccttcatcatggcagaacctgatgggtccaatcgaacagtgtttgtcagcatcgacataggcatggtatcacaaaggctcaggctggaggtcctgaacagactgcagagtaaatatggctccctgtacagaagagataatgtcatcctgagtggcactcacactcattcaggtcctgcaggatatttccagtataccgtgtttgtaattgccagtgaaggatttagcaatcaaacttttcagcacatggtcactggtatcttgaagagcattgacatagcacacacaaatatgaaaccaggcaaaatcttcatcaataaaggaaatgtggatggtgtgcagatcaacagaagtccgtattcttaccttcaaaatccgcagtcagagagagcaaggtattcttcaaatacagacaaggaaatgatagttttgaaaatggtagatttgaatggagatgacttgggccttatcagctggtttgccatccacccggtcagcatgaacaacagtaaccatcttgtaaacagtgacaatgtgggctatgcatcttacctgcttgagcaagagaagaacaaaggatatctacctggacaggggccatttgtagcagcctttgcttcatcaaacctaggagatgtgtcccccaacattcttggaccacgttgcatcaacacaggagagtcctgtgataacgccaatagcacttgtcccattggtgggcctagcatgtgcattgctaagggacctggacaggatatgtttgacagcacacaaattataggacgggccatgtatcagagagcaaagtcaaaaacatgtaaaccagcattgggctacagttttgcagctggcactattgatggagttggaggcctcaattttacacaggggaaaacagaaggggatccattttgggacaccattcgggaccagatcctgggaaagccatctgaagaaattaaagaatgtcataaaccaaagcccatccttcttcacaccggagaactatcaaaacctcacccctggcatccagacattgttgatgttcagattattacccttgggtccttggccataactgccatccccggggagtttacgaccatgtctggacgaagacttcgagaggcagttcaagcagaatttgcatctcatgggatgcagaacatgactgttgttatttcaggtctatgcaacgtctatacacattacattaccacttatgaagaataccaggctcagcgatatgaggcagcatcgacaatttatggaccgcacacattatctgcttacattcagctcttcagaaaccttgctaaggctattgctacggacacggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 10 - DNA, 501 bp, Artificial Sequence
  synthetic nucleotide
atgaggcagcatcgacaatttatggaccgcacgcattatctgcttacattcagctcttcagaaaccttgctaaggctattgctacgtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 11 - DNA, 972 bp, Artificial Sequence
  synthetic nucleotide
atggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtgaatggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttagtga

SEQ ID NO: 12 - DNA, 483 bp, Artificial Sequence
  synthetic nucleotide
atggtagccaacctgagcagaggtccagaacctccctttttcaaacaattaatagttccattaattcctagtattgtggatagagcaccaaaaggcagaactttcggggatgtcctgcagccagcaaaacctgaatacagagtgggggaagttgctgaagttatatttgtaggtgctaacccgaagaattcagtacaaaaccagacccatcagaccttcctcactgtggagaaatatgaggctacttcaacatcgtggcagatagtgtgtaatgatgcctcctgggagactcgtttttattggcacaagggactcctgggtctgagtaatgcaacagtggaatggcatattccagacactgcccagcctggaatctacagaataagatattttggacacaatcggaagcaggacattctgaagcctgctgtcatactttcatttgaaggcacttccccggcttttgaagttgtaactatttag

SEQ ID NO: 13 - DNA, 795 bp, Artificial Sequence
  synthetic nucleotide
atgcctagcatcttcgcctatcagagctccgaggtggactggtgtgagagcaacttccagtactcggagctggtggccgagttctacaacacgttctccaatatccccttcttcatcttcgggccactgatgatgctcctgatgcacccgtatgcccagaagcgctcccgctacatttacgttgtctgggtcctcttcatgatcataggcctgttctccatgtatttccacatgacgctcagcttcctgggccagctgctggacgagatcgccatcctgtggctcctgggcagtggctatagcatatggatgccccgctgctatttcccctccttccttggggggaacaggtcccagttcatccgcctggtcttcatcaccactgtggtcagcacccttctgtccttcctgcggcccacggtcaacgcctacgccctcaacagcattgccctgcacattctctacatcgtgtgccaggagtacaggaagaccagcaataaggagcttcggcacctgattgaggtctccgtggttttatgggctgttgctctgaccagctggatcagtgaccgtctgctttgcagcttctggcagaggattcatttcttctatctgcacagcatctggcatgtgctcatcagcatcaccttcccttatggcatggtcaccatggccttggtggatgccaactatgagatgccaggtgaaaccctcaaagtccgctactggcctcgggacagttggcccgtggggctgccctacgtggaaatccggggtgatgacaaggactgctga

SEQ ID NO: 14 - DNA, 828 bp, Artificial Sequence
  synthetic nucleotide
atgggcgccccgcactggtgggaccagctgcaggctggtagctcggaggtggactggtgcgaggacaactacaccatcgtgcctgctatcgccgagttctacaacacgatcagcaatgtcttatttttcattttaccgcccatctgcatgtgcttgtttcgtcagtatgcaacatgcttcaacagtggcatctacttaatctggactcttttggttgtagtgggaattggatccgtctacttccatgcaacccttagtttcttgggtcagatgcttgatgaacttgcagtcctttgggttctgatgtgtgctttggccatgtggttccccagaaggtatctaccaaagatctttcggaatgaccggggtaggttcaaggtggtggtcagtgtcctgtctgcggttacgacgtgcctggcatttgtcaagcctgccatcaacaacatctctctgatgaccctgggagttccttgcactgcactgctcatcgcagagctaaagaggtgtgacaacatgcgtgtgtttaagctgggcctcttctcgggcctctggtggaccctggccctgttctgctggatcagtgaccgagctttctgcgagctgctgtcatccttcaacttcccctacctgcactgcatgtggcacatcctcatctgccttgctgcctacctgggctgtgtatgctttgcctactttgatgctgcctcagagattcctgagcaaggccctgtcatcaagttctggcccaatgagaaatgggccttcattggtgtcccctatgtgtccctcctgtgtgccaacaagaaatcatcagtcaagatcacgtga

SEQ ID NO: 15 - DNA, 804 bp, Artificial Sequence
  synthetic nucleotide
atggctccggccgcggaccgagagggctactggggccccacgacctccacgctggactggtgcgaggagaactactccgtgacctggtacatcgccgagttctggaatacagtgagtaacctgatcatgattatacctccaatgttcggtgcagttcagagtgttagagacggtctggaaaagcggtacattgcttcttatttagcactcacagtggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 16 - DNA, 693 bp, Artificial Sequence
  synthetic nucleotide
atggctccggccgcggaccgagagggctactggggccccacgacctccacgctggactggtgcgaggagaactactccgtgacctggtacatcgccgagttcttggtaggaatgggatcctggtgcttccacatgactctgaaatatgaaatgcagctattggatgaactcccaatgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 17 - DNA, 519 bp, Artificial Sequence
  synthetic nucleotide
atgatatacagctgttgcatatttgtgtactgcatgtttgaatgtttcaagatcaagaactcagtaaactaccatctgctttttaccttagttctattcagtttaatagtaaccacagtttaccttaaggtaaaagagccgatattccatcaggtcatgtatggaatgttggtctttacattagtacttcgatctatttatattgttacatgggtttatccatggcttagaggactgggttatacatcattgggtatatttttattgggatttttattttggaatatagataacatattttgtgagtcactgaggaactttcgaaagaaggtaccacctatcataggtattaccacacaatttcatgcatggtggcatattttaactggccttggttcctatcttcacatccttttcagtttgtatacaagaacactttacctgagatataggccaaaagtgaagtttctctttggaatctggccagtgatcctgtttgagcctctcaggaagcattga

SEQ ID NO: 18 - DNA, 1965 bp, Artificial Sequence
  synthetic nucleotide
atgaatggacaccttgaagcagaggagcagcaggaccagaggccagaccaggagctgaccgggagctggggccacgggcctaggagcaccctggtcagggctaaggccatggccccgcccccaccgccactggctgccagcaccccgctcctccatggcgagtttggctcctacccagcccgaggcccacgctttgccctcacccttacatcgcaggccctgcacatacagcggctgcgccccaaacctgaagccaggccccggggtggcctggtcccgttggccgaggtctcaggctgctgcaccctgcgaagccgcagcccctcagactcagcggcctacttctgcatctacacctaccctcggggccggcgcggggcccggcgcagagccactcgcaccttccgggcagatggggccgccacctacgaagagaaccgtgccgaggcccagcgctgggccactgccctcacctgtctgctccgaggactgccactgcccggggatggggagatcacccctgacctgctacctcggccgccccggttgcttctattggtcaatccctttgggggtcggggcctggcctggcagtggtgtaagaaccacgtgcttcccatgatctctgaagctgggctgtccttcaacctcatccagacagaacgacagaaccacgcccgggagctggtccaggggctgagcctgagtgagtgggatggcatcgtcacggtctcgggagacgggctgctccatgaggtgctgaacgggctcctagatcgccctgactgggaggaagctgtgaagatgcctgtgggcatcctcccctgcggctcgggcaacgcgctggccggagcagtgaaccagcacgggggatttgagccagccctgggcctcgacctgttgctcaactgctcactgttgctgtgccggggtggtggccacccactggacctgctctccgtgacgctggcctcgggctcccgctgtttctccttcctgtctgtggcctggggcttcgtgtcagatgtggatatccagagcgagcgcttcagggccttgggcagtgcccgcttcacactgggcacggtgctgggcctcgccacactgcacacctaccgcggacgcctctcctacctccccgccactgtggaacctgcctcgcccacccctgcccatagcctgcctcgtgccaagtcggagctgaccctaaccccagacccagccccgcccatggcccactcacccctgcatcgttctgtgtctgacctgcctcttcccctgccccagcctgccctggcctctcctggctcgccagaacccctgcccatcctgtccctcaacggtgggggcccagagctggctggggactggggtggggctggggatgctccgctgtccccggacccactgctgtcttcacctcctggctctcccaaggcagctctacactcacccgtctccgaaggggcccccgtaattcccccatcctctgggctcccacttcccacccctgatgcccgggtaggggcctccacctgcggcccgcccgaccacctgctgcctccgctgggcaccccgctgcccccagactgggtgacgctggagggggactttgtgctcatgttggccatctcgcccagccacctaggcgctgacctggtggcagctccgcatgcgcgcttcgacgacggcctggtgcacctgtgctgggtgcgtagcggcatctcgcgggctgcgctgctgcgccttttcttggccatggagcgtggtagccacttcagcctgggctgtccgcagctgggctacgccgcggcccgtgccttccgcctagagccgctcacaccacgcggcgtgctcacagtggacggggagcaggtggagtatgggccgctacaggcacagatgcaccctggcatcggtacactgctcactgggcctcctggctgcccggggcgggagccctga

SEQ ID NO: 19 - DNA, 22 bp, Artificial Sequence
  synthetic nucleotide
acaggattcaaaccaggactgt

SEQ ID NO: 20 - DNA, 20 bp, Artificial Sequence
  synthetic nucleotide
tgggcatctttccttccgaa

SEQ ID NO: 21 - DNA, 22 bp, Artificial Sequence
  synthetic nucleotide
tgacaggattcaaaccaggact

SEQ ID NO: 22 - DNA, 20 bp, Artificial Sequence
  synthetic nucleotide
ctgggcatctttccttccga

SEQ ID NO: 23 - DNA, 22 bp, Artificial Sequence
  synthetic nucleotide
atactcaccgaacggaagaacc

SEQ ID NO: 24 - DNA, 22 bp, Artificial Sequence
  synthetic nucleotide
ccattagcccattcaccacctc

SEQ ID NO: 25 - DNA, 21 bp, Artificial Sequence
  synthetic nucleotide
actgatactcaccgaacggaa

SEQ ID NO: 26 - DNA, 21 bp, Artificial Sequence
  synthetic nucleotide
cattagcccattcaccacctc

SEQ ID NO: 27 - DNA, 20 bp, Artificial Sequence
  synthetic nucleotide
cacagccaacagtctccaaa

SEQ ID NO: 28 - DNA, 19 bp, Artificial Sequence
  synthetic nucleotide
tctgagtataagccgccca

SEQ ID NO: 29 - DNA, 19 bp, Artificial Sequence
  synthetic nucleotide
atagaccgagcacagccaa

SEQ ID NO: 30 - DNA, 21 bp, Artificial Sequence
  synthetic nucleotide
gaaccttctcaggattgaggt

SEQ ID NO: 31 - DNA, 21 bp, Artificial Sequence
  synthethic nucleotide
taacgaacgagactctggcat

SEQ ID NO: 32 - DNA, 22 bp, Artificial Sequence
  synthetic nucleotide
cggacatctaagggcatcacag